(19) United States
(12) Patent Application Publication
(10) Pub. No.: US 2026/0294949 A1
(43) Pub. Date: Oct. 1, 2026

(54) SUBSTITUTED BICYCLIC HETEROCYCLE COMPOUNDS, COMPOSITIONS, AND USES THEREOF

(71) Applicant: Lakewood Amedex, Inc., University Park, FL (US)

(72) Inventors: Kelvin Cooper, Sarasota, FL (US); Steven A. Kates, Needham, MA (US)

(21) Appl. No.: 19/478,661

(22) PCT Filed: Apr. 26, 2024
(86) PCT No.: PCT/US2024/026372

§ 371 Date: Oct. 24, 2025

Related U.S. Application Data
(60) Provisional application No. 63/498,469, filed on Apr. 26, 2023.

(51) Int. Cl. A61K 31/7076; A61K 31/7064; A61K 31/7068; A61K 31/708; A61P 31/04; C07H 19/207; C07H 19/23

(52) U.S. Cl. CPC A61K 31/7076; A61K 31/7064; A61K 31/7068; A61K 31/708; A61P 31/04; C07H 19/207; C07H 19/23

(57) ABSTRACT

Substituted bicyclic heterocycle compounds of Formula (I) and methods of use are disclosed.

### CROSS-REFERENCE TO RELATED APPLICATION

This application claims priority to U.S. Provisional Patent Application Ser. No. 63/498,469 filed on Apr. 26, 2023, which is incorporated by reference herein in its entirety.

### BACKGROUND

A continuing need exists for antimicrobial and antiviral compounds.

### SUMMARY

The present disclosure provides a compound having the formula:

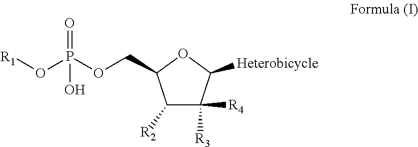

or a pharmaceutically acceptable salt thereof. R₁ is (C₁-C₈)straight-chain alkyl, (C₃-C₈)branched-chain alkyl, (C₃-C₈)cycloalkyl, phenylalkyl, alkylcycloalkyl, hydroxy(C₁-C₈)straight-chain alkyl, hydroxy(C₃-C₈)branched-chain alkyl, alkoxy(C₁-C₈)straight-chain alkyl, alkoxy(C₃-C₈)branched-chain alkyl, halo(C₁-C₈)straight-chain alkyl, or halo(C₃-C₈)branched-chain alkyl. R₂, R₃, and R₄ are each independently H, OH, oxy(C₁-C₈)straight-chain alkyl, oxy(C₁-C₈)branched-chain alkyl, CF₃, or F. Heterobicycle is selected from the group consisting of

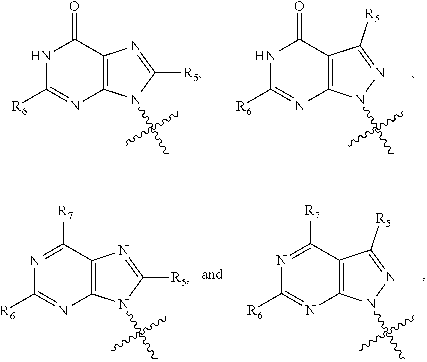

R₅ is H, (C₁-C₅)straight-chain alkyl, (C₁-C₅)branched-chain alkyl, or CF₃. R₆ and R₇ are each independently selected from H, N(R₈)R₉, (C₁-C₅)straight-chain alkyl, (C₁-C₅)branched-chain alkyl, or CF₃. R₈ and R₉ are each independently selected from H, (C₁-C₅)straight-chain alkyl, (C₁-C₅)branched-chain alkyl, or are bound to one another through a C—C bond to form a C₃-C₈ cycloalkyl.

In some embodiments, R₁ is octyl. In some embodiments, R₁ is hexyl. In some embodiments, R₁ is butyl. In some embodiments, R₁ is 2-methylpropyl. In some embodiments, R₁ is 2-methylbutyl. In some embodiments, R₁ is 1-butanolyl.

In some embodiments, R₂ is H. In some embodiments, R₂ is OH.

In some embodiments, R₃ and R₄ are both H. In some embodiments, R₃ and R₄ are both F.

In some embodiments, R₃ is OCH₃ and R₄ is H. In some embodiments, R₃ is H and R₄ is OCH₃.

In some embodiments, Heterobicycle is

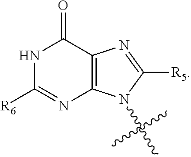

In some embodiments, R₅ is H and R₆ is NH₂. In some embodiments, R₃ is OCH₃.

In some embodiments, Heterobicycle is

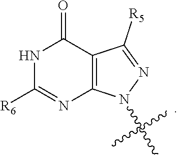

In some embodiments, Heterobicycle is

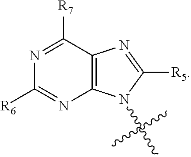

In some embodiments, R₅ and R₆ are both H and R₇ is NH₂. In some embodiments, R₅ and R₇ are both H and R₆ is NH₂.

In some embodiments, Heterobicycle is

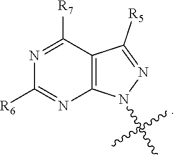

In some embodiments, R₅ and R₆ are both H and R₇ is NH₂.

In some embodiments, the compound is a compound having the formula:

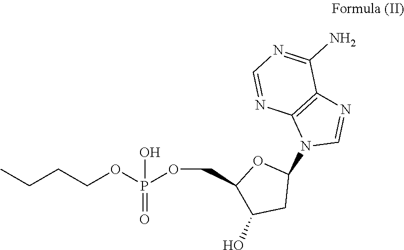 [SMILES: CCCCOP(=O)(O)OC[C@H]1O[C@@H](n2cnc3c(N)ncnc32)C[C@@H]1O]

or a pharmaceutically acceptable salt thereof.

In some embodiments, the compound is a compound having the formula:

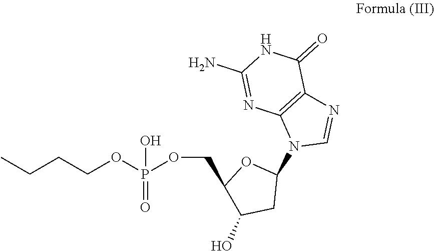 [SMILES: CCCCOP(=O)(O)OC[C@H]1O[C@@H](n2cnc3c(=O)[nH]c(N)nc32)C[C@@H]1O]

or a pharmaceutically acceptable salt thereof.

In some embodiments, the compound is a compound having the formula:

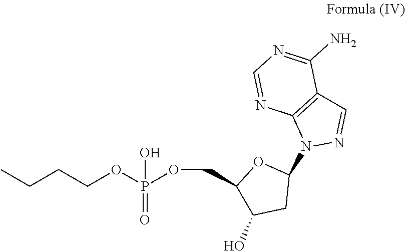 [SMILES: CCCCOP(=O)(O)OC[C@H]1O[C@@H](n2ncc3c(N)ncnc32)C[C@@H]1O]

or a pharmaceutically acceptable salt thereof.

In some embodiments, the compound is a compound having the formula:

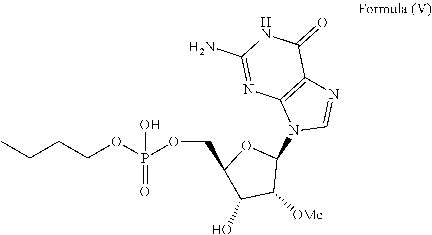 [SMILES: CCCCOP(=O)(O)OC[C@H]1O[C@@H](n2cnc3c(=O)[nH]c(N)nc32)[C@H](OC)[C@@H]1O]

or a pharmaceutically acceptable salt thereof.

In some embodiments, the compound is a compound having the formula:

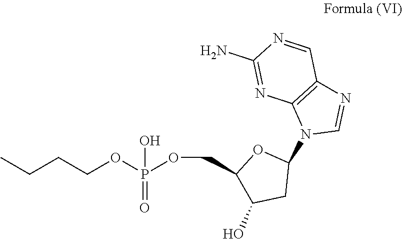 [SMILES: CCCCOP(=O)(O)OC[C@H]1O[C@@H](n2cnc3cnc(N)nc32)C[C@@H]1O]

or a pharmaceutically acceptable salt thereof.

In some embodiments, a pharmaceutical composition includes any compound disclosed herein or a pharmaceutically acceptable salt thereof, and one or more pharmaceutically acceptable carriers, excipients, diluents, or a mixture of two or more thereof.

In some embodiments, a method of treating an infection in a patient in need thereof includes administering an effective amount of any compound disclosed herein or a pharmaceutically acceptable salt thereof, to the patient.

In some embodiments, a method of treating an infection of at least one of a wound or an ulcer in a patient in need thereof includes administering an effective amount of any compound disclosed herein or a pharmaceutically acceptable salt thereof, to the patient.

In some embodiments, a method of treating an infection of a diabetic foot ulcer in a patient in need thereof includes administering an effective amount of any compound disclosed herein or a pharmaceutically acceptable salt thereof, to the patient.

In some embodiments, a method of treating a bladder and/or a urinary tract infection in a patient in need thereof includes administering an effective amount of any compound disclosed herein or a pharmaceutically acceptable salt thereof, to the patient.

In some embodiments, a method of treating a lung infection in a patient in need thereof includes administering an effective amount of any compound disclosed herein or a pharmaceutically acceptable salt thereof, to the patient. In some embodiments, the lung infection arises from a pulmonary condition. In some embodiments, the pulmonary condition is selected from the group consisting of genetic conditions, acquired conditions, primary conditions, secondary conditions, asthma, chronic obstructive pulmonary disease, cystic fibrosis, bronchiolitis, pneumonia, bronchitis, emphysema, adult respiratory distress syndrome, allergies, lung cancer, small cell lung cancer, primary lung cancer, metastatic lung cancer, bronchiestasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, and conditions caused by any one or combination of the above.

In some embodiments, the pulmonary condition arises from a coronavirus. In some embodiments, the coronavirus is selected from the group consisting of an alphacoronavirus, a betacoronavirus, a gammacoronavirus, a deltacoronavirus, and an omicroncoronavirus, or combinations thereof.

In some embodiments, the coronavirus is selected from the group consisting of porcine epidemic diarrhea virus (PEDv), scotophilus bat coronavirus 512, bat coronavirus CDPHE15, BtRF-Alpha-CoV HuB-2013, bat coronavirus HKU10, miniopterus bat coronavirus HKU8, miniopterus bat coronavirus 1, Nyctalus velutinus alphacoronavirus SC-2013, Pipistrellus kuhlii coronavirus 3398, Myotis ricketti alphacoronavirus Sax-2011, HumCoV 229E, 229E-related bat coronavirus, camel alphacoronavirus, alpaca respiratory coronavirus, HumCoV NL63, NL63-related Bat-CoV BtKYNL63-9b, HKU2, SADSr-CoV, SADS-CoV, Lucheng Rn rat coronavirus2, FIPV, TGEV, PRCV, alphacoronavirus 1, mink coronavirus 1, FRCoV-NL-2010, Sorex araneus coronavirus T14, Suncus murinus coronavirus X74, HumCoV OC43, HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYNO2, human SARS-CoV-2, cat-CoV, tiger-CoV, dog-CoV, mink-CoV, GD pangolin-CoV, bat Hp-betacoronavirus Zhejiang2013, peafowl IBV, avian coronavirus 9203, fowl IBV, avian coronavirus, duck-CoV, partridge IBV, goose coronavirus CB17, beluga whale coronavirus SW1, HKU22, HKU19, HKU20, HKU21, HKU11, HKU12, HKU16, HKU27, HKU28, HKU29, HKU18, HKU13, HKU30-UAE, HKU30-Poland, HKU17-USA, HKU17-China, AlCCoV, and HKU15, or combinations thereof.

In some embodiments, the coronavirus is a betacoronavirus selected from the group consisting of HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYNO2, human SARS-CoV-2, cat-CoV, tiger-CoV, dog-CoV, mink-CoV, GD pangolin-CoV, and bat Hp-betacoronavirus Zhejiang2013, or combinations thereof.

In some embodiments, a method of treating cystic fibrosis in a patient in need thereof includes administering an effective amount of any compound disclosed herein or a pharmaceutically acceptable salt thereof, to the patient.

In some embodiments, a method of treating pneumonia in a patient in need thereof includes administering an effective amount of any compound disclosed herein or a pharmaceutically acceptable salt thereof, to the patient. In some embodiments, the pneumonia is ventilator acquired pneumonia.

In some embodiments, a method of treating an infection in a burn wound in a patient in need thereof includes administering an effective amount of any compound disclosed herein or a pharmaceutically acceptable salt thereof, to the patient.

In some embodiments, a method of treating otitis externa in a patient in need thereof includes administering an effective amount of any compound disclosed herein or a pharmaceutically acceptable salt thereof, to the patient.

In some embodiments, a method of treating bacterial vaginosis in a patient in need thereof includes administering an effective amount of any compound disclosed herein or a pharmaceutically acceptable salt thereof, to the patient.

In some embodiments, a method of treating impetigo in a patient in need thereof includes administering an effective amount of any compound disclosed herein or a pharmaceutically acceptable salt thereof, to the patient.

In some embodiments, a method of treating oral mucositis in a patient in need thereof includes administering an effective amount of any compound disclosed herein or a pharmaceutically acceptable salt thereof, to the patient.

### DETAILED DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

The present disclosure provides a compound having the formula:

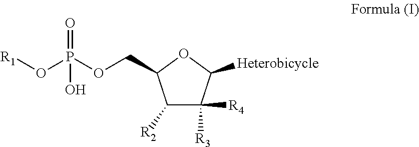

or a pharmaceutically acceptable salt thereof, wherein
R₁ is (C₁-C₈)straight-chain alkyl, (C₃-C₈)branched-chain alkyl, (C₃-C₈)cycloalkyl, phenylalkyl, alkylcycloalkyl, hydroxy(C₁-C₈)straight-chain alkyl, hydroxy(C₃-C₈)branched-chain alkyl, alkoxy(C₁-C₈)straight-chain alkyl, alkoxy(C₃-C₈)branched-chain alkyl, halo(C₁-C₈)straight-chain alkyl, or halo(C₃-C₈)branched-chain alkyl; R₂, R₃, and R₄ are each independently H, OH, oxy(C₁-C₈)straight-chain alkyl, oxy(C₁-C₈)branched-chain alkyl, CF₃, or F;
Heterobicycle is selected from the group consisting of

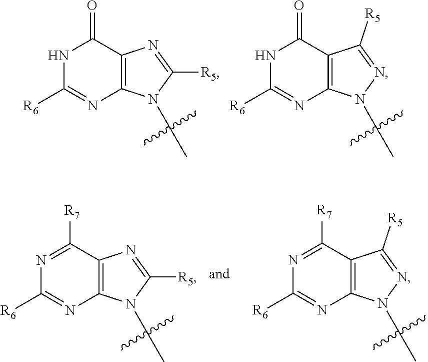

R₅ is H, (C₁-C₅)straight-chain alkyl, (C₁-C₅)branched-chain alkyl, or CF₃; R₆ and R₇ are each independently selected from H, N(R₈)R₉, (C₁-C₅)straight-chain alkyl, (C₁-C₅)branched-chain alkyl, or CF₃; and
R₈ and R₉ are each independently selected from H, (C₁-C₅)straight-chain alkyl, (C₁-C₅)branched-chain alkyl, or are bound to one another through a C—C bond to form a C₃-C₈ cycloalkyl.

In some embodiments, R₁ is a (C₁-C₈)straight-chain alkyl. In some embodiments, the straight alkyl chain is methyl:

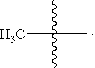

In some embodiments, R₁ is a (C₁-C₈)straight-chain alkyl. In some embodiments, the straight alkyl chain is ethyl:

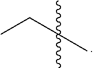 [SMILES: *CC]

In some embodiments, R₁ is a (C₁-C₈)straight-chain alkyl. In some embodiments, the straight alkyl chain is propyl:

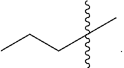 [SMILES: *CCC]

In some embodiments, R₁ is a (C₁-C₈)straight-chain alkyl. In some embodiments, the straight alkyl chain is butyl:

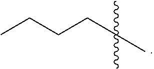 [SMILES: *CCCC]

In some embodiments, R₁ is a (C₁-C₈)straight-chain alkyl. In some embodiments, the straight alkyl chain is pentyl:

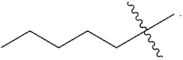 [SMILES: *CCCCC]

In some embodiments, R₁ is a (C₁-C₈)straight-chain alkyl. In some embodiments, the straight alkyl chain is hexyl:

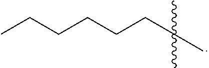 [SMILES: *CCCCCC]

In some embodiments, R₁ is a (C₁-C₈)straight-chain alkyl. In some embodiments, the straight alkyl chain is heptyl:

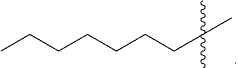 [SMILES: *CCCCCCC]

In some embodiments, R₁ is a (C₁-C₈)straight-chain alkyl. In some embodiments, the straight alkyl chain is octyl:

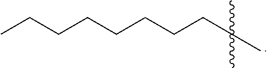 [SMILES: *CCCCCCCC]

In some embodiments, R₁ is a (C₃-C₈)branched-chain alkyl. In some embodiments, the branched alkyl chain is 2-methylpropyl:

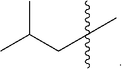 [SMILES: *CC(C)C]

In some embodiments, R₁ is a (C₃-C₈)branched-chain alkyl. In some embodiments, the branched alkyl chain is 2-methylbutyl:

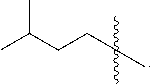 [SMILES: *CCC(C)C]

In some embodiments, R₁ is a (C₃-C₈)cycloalkyl. In some embodiments, the cycloalkyl is cyclopropyl:

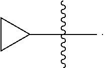 [SMILES: *C1CC1]

In some embodiments, R₁ is a (C₃-C₈)cycloalkyl. In some embodiments, the cycloalkyl is cyclobutyl:

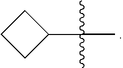 [SMILES: *C1CCC1]

In some embodiments, R₁ is a (C₃-C₈)cycloalkyl. In some embodiments, the cycloalkyl is cyclopentyl:

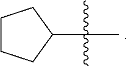 [SMILES: *C1CCCC1]

In some embodiments, R₁ is a (C₃-C₈)cycloalkyl. In some embodiments, the cycloalkyl is cyclohexyl:

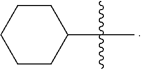 [SMILES: *C1CCCCC1]

In some embodiments, R₁ is a (C₃-C₈)cycloalkyl. In some embodiments, the cycloalkyl is cycloheptyl:

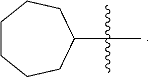 [SMILES: *C1CCCCCC1]

In some embodiments, R₁ is a (C₃-C₈)cycloalkyl. In some embodiments, the cycloalkyl is cyclooctyl:

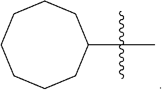 [SMILES: *C1CCCCCCC1]

In some embodiments, R₁ is aryl. In some embodiments, the aryl is phenyl:

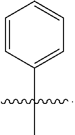 [SMILES: *c1ccccc1]

In some embodiments, R₁ is arylalkyl. In some embodiments, the arylalkyl is benzyl:

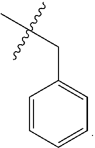 [SMILES: *Cc1ccccc1]

In some embodiments, R₁ is alkylcycloalkyl. In some embodiments, the alkylcycloalkyl is methylcyclohexyl:

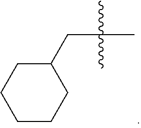 [SMILES: *CC1CCCCC1]

In some embodiments, R₁ is alkylcycloalkyl. In some embodiments, the alkylcycloalkyl is ethylcyclohexyl:

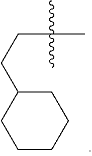 [SMILES: *CCC1CCCCC1]

In some embodiments, R₁ is hydroxy(C₁-C₈)straight-chain alkyl. In some embodiments, the straight-chain alkanol is 1-butanolyl:

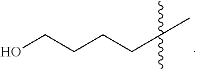 [SMILES: *CCCCO]

In some embodiments, R₁ is hydroxy(C₃-C₈)branched-chain alkyl. In some embodiments, the branched-chain alkanol is 3-methyl-1-butanol-3-yl:

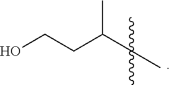 [SMILES: *C(C)CCO]

In some embodiments, R₁ is alkoxy(C₁-C₈)straight-chain alkyl. In some embodiments, the straight-chain alkoxyalkyl is ethoxyethyl:

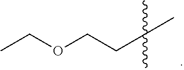 [SMILES: *CCOCC]

In some embodiments, R₁ is alkoxy(C₃-C₈)branched-chain alkyl. In some embodiments, the branched-chain alkoxyalkyl is isopropxyethyl:

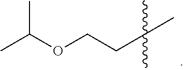 [SMILES: *CCOC(C)C]

In some embodiments, R₁ is a halo(C₁-C₈)straight-chain alkyl. In some embodiments, the straight-chain haloalkyl is 1,1,1-trifluorobutyl:

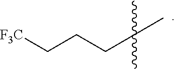 [SMILES: *CCCC(F)(F)F]

In some embodiments, R₁ is a halo(C₃-C₈)branched-chain alkyl. In some embodiments, the branched-chain haloalkyl is 2-trifluoromethylpentyl:

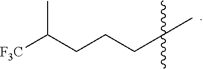 [SMILES: *CCCC(C)C(F)(F)F]

In some embodiments, R₂ is H. In some embodiments, R₂ is OH.

In some embodiments, R₃ is H. In some embodiments, R₃ is F. In some embodiments, R₃ is oxy(C₁-C₈)straight-chain alkyl. In some embodiments, R₃ is methoxy (OCH₃).

In some embodiments, R₄ is H. In some embodiments, R₄ is F.

In some embodiments, R₃ and R₄ are both H. In some embodiments, R₃ and R₄ are both F.

In some embodiments, R₃ is OCH₃ and R₄ is H. In some embodiments, R₃ is H and R₄ is OCH₃.

In some embodiments, Heterobicycle is a substituted bicyclic heterocycle selected from the group consisting of

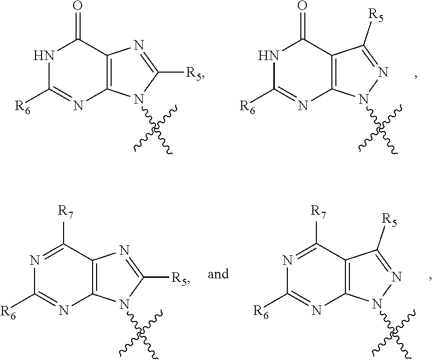

in which R₅ is H, (C₁-C₅)straight-chain alkyl, (C₁-C₅)branched-chain alkyl, or CF₃; R₆ and R₇ are each independently selected from H, N(R₈)R₉, (C₁-C₅)straight-chain alkyl, (C₁-C₅)branched-chain alkyl, or CF₃; and R₈ and R₉ are each independently selected from H, (C₁-C₅)straight-chain alkyl, (C₁-C₅)branched-chain alkyl, or are bound to one another through a C—C bond to form a C₃-C₈ cycloalkyl.

In embodiments, Heterobicycle is

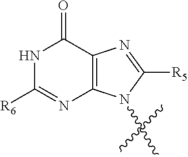

and R₅ is H and R₆ is NH₂. In further embodiments, R₃ is OCH₃.

In embodiments, Heterobicycle is

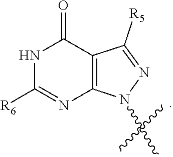

In embodiments, Heterobicycle is

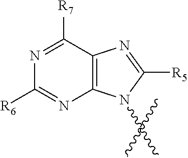

and R₅ and R₆ are both H and R₇ is NH₂. In further embodiments, R₅ and R₇ are both H and R₆ is NH₂.

In embodiments, Heterobicycle is

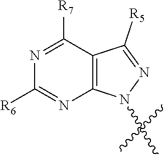

and R₅ and R₆ are both H and R₇ is NH₂.

The present disclosure also provides the compounds as described in TABLE 1. As used herein, the term “Cmpd” refers to the compound of Formula (I), or a pharmaceutically acceptable salt thereof, where R₁ to R₆ are defined as R₁ to R₆ in one of the rows of TABLE 1 labeled Cmpd 1 to Cmpd 5. For example, in the present disclosure or the claims, “Cmpd 1 as described in TABLE 1” refers specifically to the compound of Formula (I), or a pharmaceutically acceptable salt thereof, where R₁ to R₄ and Heterobicycle of the compound of Formula (I), or a pharmaceutically acceptable salt thereof, are as follows:

| Cmpd | R₁ | R₂ | R₃ | R₄ | Heterobicycle |
|---|---|---|---|---|---|
| 1 | 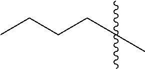 [SMILES: *CCCC] | OH | H | H | 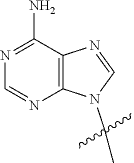 [SMILES: *n1cnc2c(N)ncnc21] |

Cmpds 1 to 5 may be made by referencing the compound of Formula (I), TABLE 1, and the synthesis schemes described in EXAMPLES 1 to 5.

TABLE 1

| Cmpd | R₁ | R₂ | R₃ | R₄ | Heterobicycle |
|---|---|---|---|---|---|
| 1 | 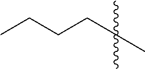 [SMILES: *CCCC] | OH | H | H | 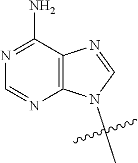 [SMILES: *n1cnc2c(N)ncnc21] |
| 2 | 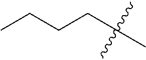 [SMILES: *CCCC] | OH | H | H | 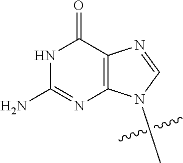 |
| 3 | 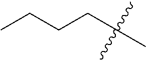 [SMILES: *CCCC] | OH | H | H | 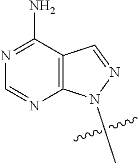 |
| 4 | 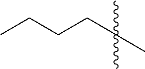 [SMILES: *CCCC] | OH | OCH₃ | H | 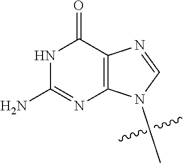 |
| 5 | 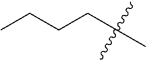 [SMILES: *CCCC] | OH | H | H | 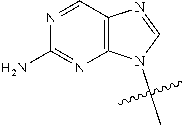 [SMILES: *n1cnc2cnc(N)nc21] |

In some embodiments, the present disclosure also provides a compound having the formula:

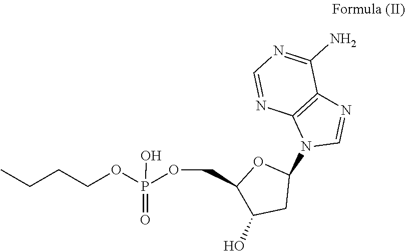 [SMILES: CCCCOP(=O)(O)OC[C@H]1O[C@@H](n2cnc3c(N)ncnc32)C[C@@H]1O]

or a pharmaceutically acceptable salt thereof.

The chemical name of the compound of Formula (II) is (2R,3S,5R)-5-(6-aminopurin-9-yl)-3-hydroxytetrahydrofuran-2-methyl butyl phosphate. The molecular formula of the compound of Formula (II) is C₁₄H₂₂N₅O₆P. The molecular weight of the compound of Formula (II) is 387.33 Da. The compound of Formula (II) is also referred to herein as LAI-017, Compound (II) or Cmpd 1, which such terms are used interchangeably herein. In some embodiments, LAI-017 includes a 2-deoxyribose, a phosphate group, a butyl group, and an adenine.

In some embodiments, the present disclosure also provides a compound having the formula:

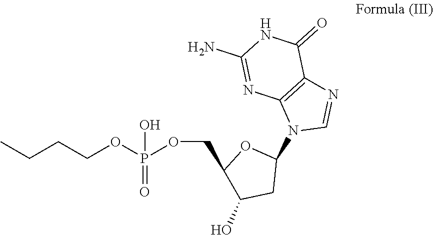 [SMILES: CCCCOP(=O)(O)OC[C@H]1O[C@@H](n2cnc3c(=O)[nH]c(N)nc32)C[C@@H]1O]

or a pharmaceutically acceptable salt thereof.

The chemical name of the compound of Formula (III) is (2R,3S,5R)-5-(2-amino-1,9-dihydro-6H-purin-6-one-9-yl)-3-hydroxytetrahydrofuran-2-methyl butyl phosphate. The molecular formula of the compound of Formula (III) is C₁₄H₂₂N₅O₇P. The molecular weight of the compound of Formula (III) is 403.33 Da. The compound of Formula (III) is also referred to herein as LAI-R-4, Compound (III) or Cmpd 2, which such terms are used interchangeably herein. In some embodiments, LAI-R-4 includes a 2-deoxyribose, a phosphate group, a butyl group, and a guanine.

In some embodiments, the present disclosure also provides a compound of the formula:

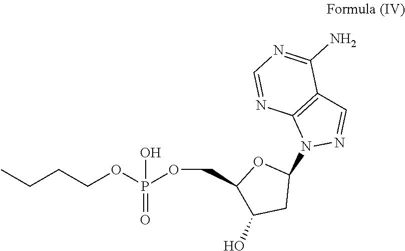 [SMILES: CCCCOP(=O)(O)OC[C@H]1O[C@@H](n2ncc3c(N)ncnc32)C[C@@H]1O]

or a pharmaceutically acceptable salt thereof.

The chemical name of the compound of Formula (IV) is (2R,3S,5R)-5-(4-amino-1-H-pyrazolo[3,4-d]pyrimidin-1-yl-3-hydroxytetrahydrofuran-2-methyl butyl phosphate. The molecular formula of the compound of Formula (IV) is C₁₄H₂₂N₅O₆P. The molecular weight of the compound of Formula (IV) is 387.33 Da. The compound of Formula (IV) is also referred to herein as LAI-018, Compound (IV) or Cmpd 3, which such terms are used interchangeably herein. In some embodiments, LAI-018 includes a 2-deoxyribose, a phosphate group, a butyl group, and a pyrazolopyrimidine.

In some embodiments, the present disclosure also provides a compound of the formula:

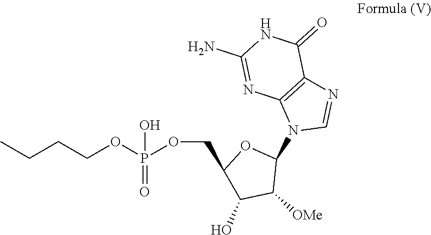 [SMILES: CCCCOP(=O)(O)OC[C@H]1O[C@@H](n2cnc3c(=O)[nH]c(N)nc32)[C@H](OC)[C@@H]1O]

or a pharmaceutically acceptable salt thereof.

The chemical name of the compound of Formula (V) is ((2R,3R,4R,5R)-5-(2-amino-1,9-dihydro-6H-purin-6-one-9-yl)-4-methoxy-3-hydroxytetrahydrofuran-2-yl)-methyl butyl hydrogen phosphate. The molecular formula of the compound of Formula (V) is C₁₅H₂₄N₅O₈P. The molecular weight of the compound of Formula (V) is 433.35 Da. The compound of Formula (V) is also referred to herein as LAI-R-8, Compound (V) or Cmpd 4, which such terms are used interchangeably herein. In some embodiments, LAI-R-8 includes a 1-methoxy-2-deoxyribose, a phosphate group, a butyl group, and a guanine.

In some embodiments, the present disclosure also provides a compound of the formula:

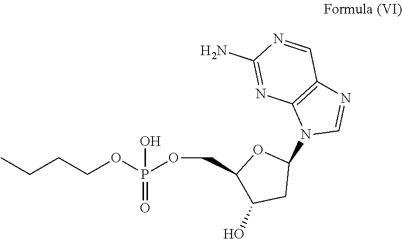 [SMILES: CCCCOP(=O)(O)OC[C@H]1O[C@@H](n2cnc3cnc(N)nc32)C[C@@H]1O]

or a pharmaceutically acceptable salt thereof.

The chemical name of the compound of Formula (VI) is ((2R,3S,5R)-5-(2-amino-9H-purin-9-yl)-3-hydroxytetrahydrofuran-2-yl)-methyl butyl hydrogen phosphate. The molecular formula of the compound of Formula (VI) is C₁₄H₂₂N₅O₆P. The molecular weight of the compound of Formula (VI) is 387.33 Da. The compound of Formula (VI) is also referred to herein as LAI-020, Compound (VI) or Cmpd 5, which such terms are used interchangeably herein. In some embodiments, LAI-020 includes a 2-deoxyribose, a phosphate group, a butanolyl group, and a 2-aminopurine.

A compound of the present disclosure is described with reference to a specific compound illustrated herein. In addition, a compound of the present disclosure may exist in any number of different forms or derivatives, all within the scope of the present disclosure. Alternative forms or derivatives, include, for example, pharmaceutically acceptable salts, prodrugs and active metabolites, tautomers, and solid forms, including without limitation different crystal forms, polymorphic or amorphous solids, including hydrates and solvates thereof, and other forms.

Unless specified to the contrary, specification of a compound of the present disclosure herein includes pharmaceutically acceptable salts of such compound. Thus, a compound of the present disclosure can be in the form of pharmaceutically acceptable salts or can be formulated as pharmaceutically acceptable salts. Contemplated pharmaceutically acceptable salt forms of the present disclosure include, without limitation, mono, bis, tris, tetrakis, and so on. Pharmaceutically acceptable salts of the present disclosure are non-toxic in the amounts and concentrations at which such pharmaceutically acceptable salts are administered. The preparation of such pharmaceutically acceptable salts of the present disclosure can facilitate the pharmacological use by altering the physical characteristics of a compound of the present disclosure without preventing it from exerting its physiological effect.

As used herein, the term “pharmaceutically acceptable,” with respect to salts and formulation components such as carriers, excipients, and diluents, refers to those salts and components which are not deleterious to a patient and which are compatible with other ingredients, active ingredients, salts or components. Pharmaceutically acceptable includes “veterinarily acceptable,” and thus includes both human and non-human mammal applications independently.

As used herein, the term “pharmaceutically acceptable salt” refers to salts commonly used to form alkali metal salts and to form addition salts of free acids or free bases. Such salts include, for example, the physiologically acceptable salts listed in Handbook of Pharmaceutical Salts: Properties, Selection and Use, P. H. Stahl and C. G. Wermuth (Eds.), Wiley-VCH, New York, 2002, which are known to the skilled artisan. Salt formation can occur at one or more positions having labile protons. The pharmaceutically acceptable salts of a compound of the present disclosure include both acid addition salts and base addition salts.

In some embodiments, suitable pharmaceutically acceptable acid addition salts of the compounds of the present disclosure may be prepared from an inorganic acid or an organic acid. Examples of such inorganic acids include without limitation hydrochloric, hydrobromic, hydroiodic, nitric, carbonic, sulfuric, and phosphoric acid. Appropriate organic acids include without limitation aliphatic, cycloaliphatic, aromatic, arylaliphatic, heterocyclic, carboxylic, and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, beta-hydroxybutyric, malonic, galactic, and galacturonic acid, to name a few. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts, among others.

In some embodiments, suitable pharmaceutically acceptable base addition salts of the compounds of the present disclosure include without limitation metallic salts made from aluminum, calcium, lithium, magnesium, potassium, sodium and zinc, or organic salts made from N,N′-dibenzylethylene-diamine, chloroprocaine, choline, diethanolamine, ethylenediamine, N-methylglucamine, lysine, arginine, and procaine. All of these salts may be prepared by conventional means from a compound of the present disclosure by treating a compound of the present disclosure with the appropriate acid or base. Pharmaceutically acceptable basic/cationic salts also include diethanolamine, ammonium, ethanolamine, piperazine and triethanolamine salts, to name a few. In some embodiments, a pharmaceutically acceptable salt of the present disclosure comprises a monovalent cation or a divalent cation.

In some embodiments, a pharmaceutically acceptable salt of the present disclosure is selected from the group consisting of an ammonium salt, a calcium salt, a sodium salt, a potassium salt, a magnesium salt, and a cobalt salt of LAI-017. In some embodiments, the ammonium salt of LAI-017 is ammonium (2R,3S,5R)-5-(6-aminopurin-9-yl)-3-hydroxytetrahydrofuran-2-methyl butyl phosphate. In some embodiments, the calcium salt of LAI-017 is calcium (2R,3S,5R)-5-(6-aminopurin-9-yl)-3-hydroxytetrahydrofuran-2-methyl butyl phosphate. In some embodiments, the sodium salt of LAI-017 is sodium (2R,3S,5R)-5-(6-aminopurin-9-yl)-3-hydroxytetrahydrofuran-2-methyl butyl phosphate. In some embodiments, the potassium salt of LAI-017 is potassium (2R,3S,5R)-5-(6-aminopurin-9-yl)-3-hydroxytetrahydrofuran-2-methyl butyl phosphate. In some embodiments, the magnesium salt of LAI-017 is magnesium (2R,3S,5R)-5-(6-aminopurin-9-yl)-3-hydroxytetrahydrofuran-2-methyl butyl phosphate. In some embodiments, the cobalt salt of LAI-017 is cobalt (2R,3S,5R)-5-(6-aminopurin-9-yl)-3-hydroxytetrahydrofuran-2-methyl butyl phosphate.

In some embodiments, a pharmaceutically acceptable salt of the present disclosure is selected from the group consisting of an ammonium salt, a calcium salt, a sodium salt, a potassium salt, a magnesium salt, and a cobalt salt of LAI-R-4. In some embodiments, the ammonium salt of LAI-R-4 is ammonium (2R,3S,5R)-5-(2-amino-1,9-dihydro-6H-purin-6-one-9-yl)-3-hydroxytetrahydrofuran-2-methyl butyl phosphate. In some embodiments, the calcium salt of LAI-R-4 is calcium (2R,3S,5R)-5-(2-amino-1,9-dihydro-6H-purin-6-one-9-yl)-3-hydroxytetrahydrofuran-2-methyl butyl phosphate. In some embodiments, the sodium salt of LAI-R-4 is sodium (2R,3S,5R)-5-(2-amino-1,9-dihydro-6H-purin-6-one-9-yl)-3-hydroxytetrahydrofuran-2-methyl butyl phosphate. In some embodiments, the potassium salt of LAI-R-4 is potassium (2R,3S,5R)-5-(2-amino-1,9-dihydro-6H-purin-6-one-9-yl)-3-hydroxytetrahydrofuran-2-methyl butyl phosphate. In some embodiments, the magnesium salt of LAI-R-4 is magnesium (2R,3S,5R)-5-(2-amino-1,9-dihydro-6H-purin-6-one-9-yl)-3-hydroxytetrahydrofuran-2-methyl butyl phosphate. In some embodiments, the cobalt salt of LAI-R-4 is cobalt (2R,3S,5R)-5-(2-amino-1,9-dihydro-6H-purin-6-one-9-yl)-3-hydroxytetrahydrofuran-2-methyl butyl phosphate.

In some embodiments, a pharmaceutically acceptable salt of the present disclosure is selected from the group consisting of an ammonium salt, a calcium salt, a sodium salt, a potassium salt, a magnesium salt, and a cobalt salt of LAI-018. In some embodiments, the ammonium salt of LAI-018 is ammonium (2R,3S,5R)-5-(4-amino-1-H-pyrazolo[3,4-d]pyrimidin-1-yl-3-hydroxytetrahydrofuran-2-methyl butyl phosphate. In some embodiments, the calcium salt of LAI-018 is calcium (2R,3S,5R)-5-(4-amino-1-H-pyrazolo[3,4-d]pyrimidin-1-yl-3-hydroxytetrahydrofuran-2-methyl butyl phosphate. In some embodiments, the sodium salt of LAI-018 is sodium (2R,3S,5R)-5-(4-amino-1-H-pyrazolo[3,4-d]pyrimidin-1-yl-3-hydroxytetrahydrofuran-2-methyl butyl phosphate. In some embodiments, the potassium salt of LAI-018 is potassium (2R,3S,5R)-5-(4-amino-1-H-pyrazolo[3,4-d]pyrimidin-1-yl-3-hydroxytetrahydrofuran-2-methyl butyl phosphate. In some embodiments, the magnesium salt of LAI-018 is magnesium (2R,3S,5R)-5-(4-amino-1-H-pyrazolo[3,4-d]pyrimidin-1-yl-3-hydroxytetrahydrofuran-2-methyl butyl phosphate. In some embodiments, the cobalt salt of LAI-018 is cobalt (2R,3S,5R)-5-(4-amino-1-H-pyrazolo[3,4-d]pyrimidin-1-yl-3-hydroxytetrahydrofuran-2-methyl butyl phosphate.

In some embodiments, a pharmaceutically acceptable salt of the present disclosure is selected from the group consisting of an ammonium salt, a calcium salt, a sodium salt, a potassium salt, a magnesium salt, and a cobalt salt of LAI-R-8. In some embodiments, the ammonium salt of LAI-R-8 is ammonium ((2R,3R,4R,5R)-5-(2-amino-1,9-dihydro-6H-purin-6-one-9-yl)-4-methoxy-3-hydroxytetrahydrofuran-2-yl)-methyl butyl phosphate. In some embodiments, the calcium salt of LAI-R-8 is calcium ((2R,3R,4R,5R)-5-(2-amino-1,9-dihydro-6H-purin-6-one-9-yl)-4-methoxy-3-hydroxytetrahydrofuran-2-yl)-methyl butyl phosphate. In some embodiments, the sodium salt of LAI-R-8 is sodium ((2R,3R,4R,5R)-5-(2-amino-1,9-dihydro-6H-purin-6-one-9-yl)-4-methoxy-3-hydroxytetrahydrofuran-2-yl)-methyl butyl phosphate. In some embodiments, the potassium salt of LAI-R-8 is potassium ((2R,3R,4R,5R)-5-(2-amino-1,9-dihydro-6H-purin-6-one-9-yl)-4-methoxy-3-hydroxytetrahydrofuran-2-yl)-methyl butyl phosphate. In some embodiments, the magnesium salt of LAI-R-8 is magnesium ((2R,3R,4R,5R)-5-(2-amino-1,9-dihydro-6H-purin-6-one-9-yl)-4-methoxy-3-hydroxytetrahydrofuran-2-yl)-methyl butyl phosphate. In some embodiments, the cobalt salt of LAI-R-8 is cobalt ((2R,3R,4R,5R)-5-(2-amino-1,9-dihydro-6H-purin-6-one-9-yl)-4-methoxy-3-hydroxytetrahydrofuran-2-yl)-methyl butyl phosphate.

In some embodiments, a pharmaceutically acceptable salt of the present disclosure is selected from the group consisting of an ammonium salt, a calcium salt, a sodium salt, a potassium salt, a magnesium salt, and a cobalt salt of LAI-020. In some embodiments, the ammonium salt of LAI-020 is ammonium (2R,3S,5R)-5-(4-aminopurin-9-yl)-3-hydroxytetrahydrofuran-2-methyl butyl phosphate. In some embodiments, the calcium salt of LAI-020 is calcium (2R,3S,5R)-5-(4-aminopurin-9-yl)-3-hydroxytetrahydrofuran-2-methyl butyl phosphate. In some embodiments, the sodium salt of LAI-020 is sodium (2R,3S,5R)-5-(4-aminopurin-9-yl)-3-hydroxytetrahydrofuran-2-methyl butyl phosphate. In some embodiments, the potassium salt of LAI-020 is potassium (2R,3S,5R)-5-(4-aminopurin-9-yl)-3-hydroxytetrahydrofuran-2-methyl butyl phosphate. In some embodiments, the magnesium salt of LAI-020 is magnesium (2R,3S,5R)-5-(4-aminopurin-9-yl)-3-hydroxytetrahydrofuran-2-methyl butyl phosphate. In some embodiments, the cobalt salt of LAI-020 is cobalt (2R,3S,5R)-5-(4-aminopurin-9-yl)-3-hydroxytetrahydrofuran-2-methyl butyl phosphate.

Pharmaceutically acceptable salts of the present disclosure can be prepared by standard techniques known in the art to which the present disclosure pertains. For example, the free-base form of a compound of the present disclosure can be dissolved in a suitable solvent, such as an aqueous or aqueous-alcohol solution containing the appropriate acid and then isolated by evaporating the solution. In another example, a salt can be prepared by reacting the free base and acid in an organic solvent. If the particular compound is an acid, the desired pharmaceutically acceptable salt may be prepared by any suitable method, including, for example, treatment of the free acid with an appropriate inorganic or organic base.

In addition to the compounds of the present disclosure, the present disclosure also includes prodrugs (e.g., pharmaceutically acceptable prodrugs), active metabolic derivatives (active metabolites), and their pharmaceutically acceptable salts.

Prodrugs are compounds or pharmaceutically acceptable salts thereof which, when metabolized under physiological conditions or when converted by solvolysis, yield the desired active compound. Typically, the prodrug is inactive, or less active than the active compound, but may provide one or more advantageous handling, administration, and/or metabolic properties. Some prodrugs are activated enzymatically to yield the active compound, or a compound may undergo further chemical reaction to yield the active compound. Prodrugs may proceed from prodrug form to active form in a single step or may have one or more intermediate forms which such forms have activity or may be inactive.

As described in The Practice of Medicinal Chemistry, Ch. 31-32 (Ed. Wermuth, Academic Press, San Diego, Calif, 2001), prodrugs can be conceptually divided into two non-exclusive categories, including bioprecursor prodrugs and carrier prodrugs. Generally, bioprecursor prodrugs are compounds that are inactive or have low activity compared to the corresponding active drug compound that contain one or more protective groups and are converted to an active form by metabolism or solvolysis. Both the active drug form and any released metabolic products should have acceptably low toxicity. Typically, the formation of active drug compound involves a metabolic process or reaction that is one of the following types: Oxidative reactions: Oxidative reactions are exemplified without limitation by reactions such as oxidation of alcohol, carbonyl, and acid functionalities, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-dealkylation, oxidative O- and S-dealkylation, oxidative deamination, as well as other oxidative reactions.

Reductive reactions: Reductive reactions are exemplified without limitation by reactions such as reduction of carbonyl functionalities, reduction of alcohol functionalities and carbon-carbon double bonds, reduction of nitrogen-containing functional groups, and other reduction reactions.

Reactions without change in the oxidation state: Reactions without change in the state of oxidation are exemplified without limitation by reactions such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improves uptake and/or localized delivery to a site(s) of action. Desirably for such a carrier prodrug, the linkage between the drug moiety and the transport moiety is a covalent bond, the prodrug is inactive or less active than the drug compound, and the prodrug and any release transport moiety are acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins.

Such carrier prodrugs are often advantageous for orally administered drugs. In some instances, the transport moiety provides targeted delivery of the drug. For example, the drug may be conjugated to an antibody or antibody fragment. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols. Wermuth, supra.

Metabolites, e.g., active metabolites, overlap with prodrugs as described above, e.g., bioprecursor prodrugs. Thus, such metabolites are pharmacologically active compounds or compounds that further metabolize to pharmacologically active compounds that are derivatives resulting from metabolic processes in the body of a subject. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug. For example, in some compounds, one or more alkoxy groups can be metabolized to hydroxyl groups while retaining pharmacologic activity and/or carboxyl groups can be esterified, e.g., glucuronidation. In some cases, there can be more than one metabolite, where an intermediate metabolite(s) is further metabolized to provide an active metabolite. For example, in some cases a derivative compound resulting from metabolic glucuronidation may be inactive or of low activity and can be further metabolized to provide an active metabolite.

Metabolites of a compound of the present disclosure may be identified using routine techniques known in the art, and their activities determined using tests such as those described in Bertolini et al., 1997, J. Med. Chem., 40:2011-2016; Shan et al., 1997, J Pharm Sci 86(7):756-757; Bagshawe, 1995, Drug Dev. Res., 34:220-230; Wermuth, supra.

It is understood by those skilled in the art that some compounds may exhibit tautomerism. In such cases, the formulae provided herein expressly depict only one of the possible tautomeric forms. It is therefore to be understood that a compound of the present disclosure intends to represent any tautomeric form of the depicted compound and is not to be limited merely to the specific tautomeric form depicted by the drawing of the compound.

In the case of agents that are solids, it is understood by those skilled in the art that the compounds and salts may exist in different crystal or polymorphic forms, or may be formulated as co-crystals, or may be in an amorphous form, or may be any combination thereof (e.g. partially crystalline, partially amorphous, or mixtures of polymorphs) all of which are intended to be within the scope of the present disclosure and specified formulae. Whereas salts are formed by acid/base addition (i.e., a free base or free acid of the compound of interest forms an acid/base reaction with a corresponding addition base or addition acid, respectively, resulting in an ionic charge interaction), co-crystals are a new chemical species that is formed between neutral compounds, resulting in the compound and an additional molecular species in the same crystal structure.

In some instances, a compound of the present disclosure is complexed with an acid or a base, including without limitation base addition salts such as, for example, ammonium, diethylamine, ethanolamine, ethylenediamine, diethanolamine, t-butylamine, piperazine, meglumine; acid addition salts, such as, for example, acetate, acetylsalicylate, besylate, camsylate, citrate, formate, fumarate, glutarate, hydrochlorate, maleate, mesylate, nitrate, oxalate, phosphate, succinate, sulfate, tartrate, thiocyanate, and tosylate; and amino acids such as, for example, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine.

Additionally, a compound of the present disclosure is intended to cover hydrated or solvated as well as unhydrated or unsolvated forms. Other examples of solvates include without limitation a compound of the present disclosure in combination with a suitable solvent, such as isopropanol, ethanol, methanol, DMSO, ethyl acetate, acetic acid or ethanolamine, and the like.

In some embodiments, a compound of the present disclosure is a protonated compound. As used herein, the term “protonated compound” refers to a compound of the present disclosure that is protonated by adding protons (or positively charged hydrogen ions) to proton acceptor sites of a compound of the present disclosure. In some embodiments, the proton acceptor sites include the phosphate groups of a compound of the present disclosure as well as any additional proton acceptor sites on either the ribose or the butyl groups of a compound of the present disclosure.

As the number of proton acceptor sites that are protonated on a compound of the present disclosure increases, the pH obtained when a compound of the present disclosure is dissolved in water having a pH of 7 decreases and thus the amount of protonation of a compound of the present disclosure can be determined by measuring the pH of solutions of water after addition of a compound of the present disclosure. pH indicates the hydrogen ion concentration of a solution.

Solutions with a high concentration of hydrogen ions have a low pH and are therefore acidic, whereas solutions with a low concentration of hydrogen ions have a high pH and are therefore basic. In some embodiments, the compounds of the present disclosure are protonated so that when dissolved in water (pH 7) such compounds form an aqueous solution having a pH of from less than about pH 7 to about pH 1. As used herein, the term “about,” when used with numerical values is to be read as including the amount(s) specified and variations of 20%, 10%, 5%, 1%, 0.5%, and 0.1% of the amount specified. In some embodiments, a compound of the present disclosure is a protonated compound having a pH when dissolved in water of from less than about pH 6 to about pH 1. In some embodiments, a compound of the present disclosure is a protonated compound having a pH when dissolved in water of from about pH 5 to about pH 1. In some embodiments, a compound of the present disclosure is a protonated compound having a pH when dissolved in water of from about pH 4.5 to about pH 1. In some embodiments, a compound of the present disclosure is a protonated compound having a pH when dissolved in water of from about pH 4 to about pH 1. In some embodiments, a compound of the present disclosure is a protonated compound having a pH when dissolved in water of from about pH 3 to about pH 1. In some embodiments, a compound of the present disclosure is a protonated compound having a pH when dissolved in water of from about pH 2 to about pH 1. In some embodiments, a compound of the present disclosure is a protonated compound having a pH when dissolved in water of about pH 3 to about pH 5. In some embodiments, a compound of the present disclosure is a protonated compound having a pH when dissolved in water of about pH 3 to about pH 4.

In some embodiments, protonation can be accomplished by incubating a compound of the present disclosure in the presence of a strong acid. Although a compound of the present disclosure can be protonated by adding protons to the reactive sites on the compound, other modifications of a compound of the present disclosure are possible and are intended to be encompassed by the term protonated compound as used herein. In some embodiments, protonated forms of the compounds of the present disclosure can be generated by subjecting the purified, partially purified or crude compounds to a low pH (e.g., acidic) environment. In some embodiments, purified or crude compounds can be protonated with acid, including phosphoric acid, nitric acid, hydrochloric acid, and acetic acid.

Other procedures to prepare a protonated compound of the present disclosure known to the skilled artisan are equally contemplated to be within the scope of the present disclosure. In some embodiments, once the compounds of the present disclosure have been protonated, such compounds may be separated from any undesired components such as, for example, excess acid.

The skilled artisan would know of many ways to separate the compounds from undesired components, including but not limited to using an H+-cation exchanger (e.g., H+-SCX). In some embodiments, the compounds of the present disclosure may be subjected to chromatography following protonation. In some embodiments, a compound of the present disclosure is run over a poly(styrene-divinyl benzene) based resin (e.g., Hamilton's PRP-1 or 3 and Polymer Lab's PLRP) following protonation.

In some embodiments, the protonated compounds of the present disclosure can be used directly. In some embodiments, the protonated compounds of the present disclosure can be processed further to remove any excess acid or salt, e.g., via precipitation, reverse phase chromatography, diafiltration or gel filtration. In some embodiments, the protonated compounds of the present disclosure can be concentrated by lyophilization, solvent evaporation, and the like. In some embodiments, when suspended in water or saline, the compounds of the present disclosure generally exhibit a pH of from about pH 3 to about pH 5 depending upon the level of protonation/acidification, which is determined by how much acid is used in the acidification process. In some embodiments, compounds of the present disclosure can be protonated by passage over a cation exchange column charged with hydrogen ions.

In some embodiments, utilization of a butyl group in a compound of the present disclosure prevents or limits substantial nuclease degradation, including without limitation exonuclease degradation, of a compound of the present disclosure. In some embodiments, the butyl group is positioned to protect the ribose of a compound of the present disclosure. Percent acid degradation may be determined using analytical HPLC to assess the loss of functional molecules or by other suitable methods. Acid degradation is generally measured as a function of time. In some embodiments, the compounds of the present disclosure are also nuclease resistant, which allows such compounds to maintain activity (e.g., pH stability) in an in vivo setting. Percent degradation of the compounds of the present disclosure in a setting containing a nuclease may be determined by methods known to those skilled in the art, such as, for example, mass spectroscopy. Nuclease degradation is generally measured as a function of time. In some embodiments, a reference compound is employed in determining the extent or rate of acid or nuclease degradation.

A compound of the present disclosure in accordance with some embodiments is useful as an antimicrobial having activity against any microbe. As used herein, the terms “microbe,” “microbial,” and like terms refers to bacteria, fungi, protozoa, viruses, yeast, and the like. As used herein, the term “antimicrobial” refers to a compound of the present disclosure having the ability to kill or inhibit the growth of a microbe, or to attenuate the severity of a microbial infection. A non-limiting list of the bacteria that a compound of the present disclosure is effective against include without limitation gram positive bacteria, gram negative bacteria, slow growing bacteria and acid fast bacteria, and any species included in the following genera: Aerococcus, Listeria, Streptomyces, Chlamydia, Lactobacillus, Eubacterium, Burkholderia, Stentrophomonas, Achromobacter, Arachnid, Mycobacterium, Peptostreptococcus, Staphylococcus, Corynebacterium, Erysipelothrix, Dermatophilus, Rhodococcus, Pseudomonas, Streptococcus, Bacillus, Peptococcus, Pneumococcus, Micrococcus, Neisseria, Klebsiella, Kurthia, Nocardia, Serratia, Rothia, Escherichia, Propionibacterium, Actinomyces, Helicobacter, Enterococcus, Shigella, Vibrio, Clostridium, Salmonella, Yersinia, and Haemophilus.

A non-limiting list of the fungi that a compound of the present disclosure is effective against include without limitation Trichophyton, Epidermophyton, Microsporum, Candida albicans and other Candida species, Pityrosporum orbiculare, Trichophyton mentagrophytes, Trichophyton rubrum, Epidermophyton floccosurn, and Trichophyton tonsurans. A non-limiting list of the viruses that a compound of the present disclosure is effective against include without limitation envelope viruses, human immunodeficiency virus (HIV), herpes simplex virus (HSV), cytomegalovirus (CMV), Hepatitis B virus (HBV), Hepatitis C virus (HCV), coronaviruses, and influenza virus.

In some embodiments, a compound of the present disclosure is useful in both therapeutic and non-therapeutic medical applications. In some embodiments including non-therapeutic medical applications, the antimicrobial effect of a compound of the present disclosure allows use of a compound of the present disclosure for sterilization (e.g., sterilization of a patient's skin or of a surface or an object, such as, for example, a surgical instrument), or sanitization (e.g., the cleansing of a surface, instrument, as to render the surface free of undesirable concentrations of disease causing microorganisms). In some embodiments, the compounds of the present disclosure are effective in combating microbial contamination of laboratory cultures, consumables (e.g., food or beverage preparations), medical devices, hospital apparatus, or industrial processes.

Therapeutic applications of a compound of the present disclosure are described herein.

The present disclosure also provides pharmaceutical compositions. As used herein, the term “pharmaceutical composition” refers to a pharmaceutical preparation that contains a compound of the present disclosure, or a pharmaceutically acceptable salt thereof, and is suitable for administration to a patient for therapeutic purposes. As used herein, the term “patient” refers to a living organism that is treated with a compound of the present disclosure, including without limitation any mammal such as, for example, humans, other primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

In some embodiments, the composition may include at least one pharmaceutically acceptable component to provide an improved formulation of a compound of the present disclosure, including without limitation one or more pharmaceutically acceptable carriers, excipients or diluents. The carrier, excipient or diluent may take a wide variety of forms depending on the form of preparation desired for administration.

As used herein, the term “carrier” includes without limitation calcium carbonate, calcium phosphate, various sugars, such as lactose, glucose, or sucrose, types of starch, cellulose derivatives, gelatin, lipids, liposomes, nanoparticles, physiologically acceptable liquids as solvents or for suspensions, including, for example, sterile solutions of water for injection (WFI), saline solution, dextrose solution, Hank's solution, Ringer's solution, vegetable oils, mineral oils, animal oils, polyethylene glycols, liquid paraffin, and the like.

As used herein, the term “excipient” generally includes without limitation fillers, binders, disintegrants, glidants, lubricants, complexing agents, solubilizers, stabilizer, preservatives, and surfactants, which may be chosen to facilitate administration of the compound by a particular route. Suitable excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, cross-linked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil, mineral oil, polyethylene glycol (e.g., PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g., 2-hydroxypropyl-delta-cyclodextrin), polysorbates (e.g., polysorbate 80), cetrimide, TPGS (d-alpha-tocopheryl polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®), polyoxyethylene sorbitan fatty acid esters, sorbitan fatty acid ester, e.g., a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, and the like.

As the skilled artisan understands, any diluent known in the art may be utilized in accordance with the present disclosure. In some embodiments of the present disclosure, the diluent is water soluble. In some embodiments of the present disclosure, the diluent is water insoluble.

As used herein, the term “diluent” includes without limitation water, saline, phosphate buffered saline (PBS), dextrose, glycerol, ethanol, buffered sodium or ammonium acetate solution, or the like, and combinations thereof.

In some embodiments, the pharmaceutical compositions of the present disclosure include at least one additional active ingredient. As used herein, the term “active ingredient” refers to a therapeutically active compound, as well as any prodrugs thereof and pharmaceutically acceptable salts, hydrates, and solvates of the compound and the prodrugs. Additional active ingredients may be combined with a compound of the present disclosure and may be either administered separately or in the same pharmaceutical composition. The amount of additional active ingredients to be given may be determined by one skilled in the art based upon therapy with a compound of the present disclosure.

In some embodiments, the composition is a human pharmaceutical composition. As used herein, the term “human pharmaceutical composition” refers to a pharmaceutical composition intended for administration to a human.

The pharmaceutical compositions of the present disclosure are suitable for administration to a patient by any suitable means, including without limitation those means used to administer conventional antimicrobials. The pharmaceutical compositions of the present disclosure may be administered using any applicable route that would be considered by one of ordinary skill, including without limitation oral, intravenous (“IV”) injection or infusion, intravesical, subcutaneous (“SC”), intramuscular (“IM”), intraperitoneal, intradermal, intraocular, inhalation (and intrapulmonary), intranasal, transdermal, epicutaneously, subdermal, topical, mucosal, nasal, ophthalmic, impression into skin, intravaginal, intrauterine, intracervical, and rectal. Such dosage forms should allow a compound of the present disclosure to reach target cells. Other factors are well known in the art and include considerations such as toxicity and dosage forms that retard a compound or composition from exerting its effects. Techniques and formulations generally may be found in Remington: The Science and Practice of Pharmacy, 21st edition, Lippincott, Williams and Wilkins, Philadelphia, Pa., 2005.

In some embodiments, the pharmaceutical compositions of the present disclosure are adapted for topical administration. As used herein, the term “topical administration” refers to administration of a compound of the present disclosure to the skin surface of a patient so that a compound of the present disclosure passes through the skin layer. Transdermal administration and transmucosal administration are also encompassed within the term topical administration. As used herein, the term “transdermal” refers to passage of a compound of the present disclosure across at least one skin layer of a patient. As used herein, “transmucosal” refers to passage of a compound of the present disclosure across a mucous membrane of a patient. Unless otherwise stated or implied, the terms “topical administration,” “transdermal administration,” and “transmucosal administration” are used interchangeably herein.

A variety of topical delivery systems for delivering bioactive compounds to microbes in a patient are well known in the art. Such systems include without limitation lotions, creams, gels, oils, ointments, solutions, suspensions, emulsions, and the like by choice of appropriate carriers in the art. In some embodiments, the pharmaceutical composition is administered in the form of a gel including a polyhydric alcohol.

Suitable carriers include without limitation vegetable or mineral oils, white petrolatum (e.g., white soft paraffin), branched chain fats or oils, animal fats and high molecular weight alcohol (e.g., greater than C₁₂). In some embodiments, carriers are selected such that a compound of the present disclosure is soluble. In some embodiments, emulsifiers, stabilizers, humectants, and antioxidants may also be included as well as agents imparting color or fragrance, if desired.

In some embodiments, an organic solvent or co-solvent such as ethanol or propanol may be employed in the pharmaceutical compositions of the present disclosure. In some embodiments, evaporation of the solvent leaves a residue on the treated surface to inhibit reinfection. In some embodiments, penetrants appropriate to the barrier to be permeated are used. Such penetrants are generally known in the art and include without limitation bile salts and fusidic acid derivatives. In some embodiments, detergents may be used to facilitate permeation. In some embodiments, creams for topical administration are formulated from a mixture of mineral oil, self-emulsifying beeswax, and water in which mixture a compound of the present disclosure, dissolved in a small amount of solvent (e.g., an oil), is admixed. The specific topical delivery system used depends on the location of the microbes.

In some embodiments, other materials may also be added to the topical pharmaceutical compositions of the present disclosure have additional moisturizing effects and to improve the consistency of the pharmaceutical composition. Examples of such compounds include without limitation cetyl esters wax, stearyl alcohol, cetyl alcohol, glycerin, methyl paraben, propyl paraben, quaternium-15, humectants, volatile methylsiloxane fluids, and polydiorganosiloxane-polyoxyalkylene. See, e.g., U.S. Pat. Nos. 5,153,230 and 4,421,769. If it is desirable for the pharmaceutical composition to have additional cleaning effects in some embodiments, chemicals such as sodium lauryl sulfate or a metal salt of a carboxylic acid may be added.

In some embodiments, a wide variety of nonvolatile emollients are useful in the pharmaceutical compositions of the present disclosure. Non-limiting examples of such nonvolatile emollients are listed in McCutcheon's, Vol. 2 Functional Materials, North American Edition, (1992), pp. 137-168, and CTFA Cosmetic Ingredient Handbook, Second Edition (1992) which lists Skin-Conditioning Agents at pp. 572-575 and Skin Protectants at p. 580. In some embodiments, the nonvolatile emollients include silicones, hydrocarbons, esters, and mixtures thereof. In some embodiments, the esters include esters of monofunctional and difunctional fatty acids that have been esterified with alcohols and polyols (i.e., alcohols having two or more hydroxyl groups). In some embodiments, long chain esters of long chain fatty acids are utilized in the pharmaceutical compositions of the present disclosure (i.e., C10-40 fatty acids esterified with C10-40 fatty alcohols). Non-limiting examples of esters useful in the pharmaceutical compositions of the present disclosure include without limitation those selected from the group consisting of diisopropyl adipate, isopropyl myristate, isopropyl palmitate, myristyl propionate, ethylene glycol distearate, 2-ethylhexyl palmitate, isodecyl neopentanoate, C12-15 alcohol benzoate, di-2-ethylhexyl maleate, ceryl palmitate, myristyl myristate, stearyl stearate, cetyl stearate, behenyl behenrate, and mixtures thereof.

Examples of silicone emollients useful in the pharmaceutical compositions of the present disclosure include without limitation polyalkylsiloxanes, cyclic polyalkylsiloxanes, and polyalkylarylsiloxanes. Suitable commercially available polyalkylsiloxanes include the polydimethylsiloxanes, which are also known as dimethicones, non-limiting examples of which include the Vicasil™ series sold by General Electric Company and the Dow Corning™ 200 series sold by Dow Corning Corporation. Commercially available polyalkylsiloxanes include cyclomethicones (Dow Corning™ 244 fluid), Dow Corning™ 344 fluid, Dow Corning™ 245 fluid and Dow Corning™ 345), among others. A suitable commercially available trimethylsiloxysilicate is sold as a mixture with dimethicone as Dow Corning™ 593 fluid. Also useful in the pharmaceutical compositions of the present disclosure are dimethiconols, which are hydroxyl terminated dimethyl silicones. Suitable commercially available dimethiconols are typically sold as mixtures with dimethicone or cyclomethicone (e.g., Dow Corning™ 1401, 1402, and 1403 fluids). Suitable commercially available polyalkylarylsiloxanes include SF1075 methylphenyl fluid (sold by General Electric Company) and 556 Cosmetic Grade phenyl trimethicone fluid (sold by Dow Corning Corporation).

Hydrocarbons suitable for use in the pharmaceutical compositions of the present disclosure include without limitation straight and branched chain hydrocarbons having from about 10 to about 30 carbon atoms. In some embodiments, the straight and branched chain hydrocarbons have from about 12 to about 24 carbon atoms. In some embodiments, the straight and branched chain hydrocarbons have from about 16 to about 22 carbon atoms. Non-limiting examples of such hydrocarbon materials include dodecane, squalane, cholesterol, 5 hydrogenated polyisobutylene, docosane (i.e., a C₂₂ hydrocarbon), hexadecane, and isohexadecane (a commercially available hydrocarbon sold as Permethyl™ 101A by Presperse, South Plainsfield, N.J.), among others.

In some embodiments, the topical pharmaceutical compositions of the present disclosure include propylene glycol. In some embodiments, propylene glycol acts as a surfactant and assists in penetration, contact, and absorption of a compound of the present disclosure. In some embodiments, propylene glycol serves as a preservative. In some embodiments, the pharmaceutical compositions of the present disclosure include a non-ionic surfactant, such as, for example, polysorbate. Such a surfactant provides better surface contact of the pharmaceutical compositions of the present disclosure with mucosa (such as vaginal mucosa) by further reducing surface tension.

The topical pharmaceutical compositions of the present disclosure optionally may also be formulated with a lipophilic phase, such as, for example, emulsions and liposome dispersions. In some embodiments, liposomal formulations may extend circulation time of a compound of the present disclosure, increase permeability of a compound of the present disclosure, and improve overall efficacy of a compound of the present disclosure as an antimicrobial. In some embodiments, a compound of the present disclosure may be combined with a lipid, cationic lipid or anionic lipid. In some embodiments, the resulting emulsion or liposomal suspension in conjunction with the pH stabilizing qualities of a compound of the present disclosure can effectively increase the in vivo half-life of the activity of a pharmaceutical composition of the present disclosure. Examples of suitable anionic lipids for use with the pharmaceutical compositions of the present disclosure include, but are not limited to, cardiolipin, dimyristoyl, dipalmitoyl, dioleoyl phosphatidyl choline, phosphatidyl glycerol, palmitoyloleoyl phosphatidyl choline, phosphatidyl glycerol, phosphatidic acid, lysophosphatidic acid, phosphatidyl serine, phosphatidyl inositol, and anionic forms of cholesterol.

In some embodiments, a compound of the present disclosure is incorporated into liposomes. In some embodiments, neutral lipids, cholesterol, and/or polyethylene glycol (PEG) are utilized in such liposomes. In some embodiments, the liposomal composition is composed of partially hydrogenated soy phosphatidylcholine (PHSC), cholesterol, methoxy-terminated PEG (mPEG), and/or distearoyl phosphatidyl ethanolamine (DSPE). The liposomes can be prepared according to any suitable method known in the art.

In some embodiments, topical administration is through nasal sprays or suppositories (rectal or vaginal). Suppositories are prepared by mixing a compound of the present disclosure with a lipid vehicle such as Theobroma oil, cacao butter, glycerin, gelatin, polyoxyethylene glycols, and the like. In some embodiments, topical administration comprises a transdermal patch or dressing such as a bandage impregnated with a compound of the present disclosure and optionally one or more carriers, excipients or diluents known in the art. In some embodiments, such dressings include without limitation semipermeable films, foams, hydrocolloids, and calcium alginate swabs. In some embodiments, the dosage administration will be continuous rather than intermittent throughout the dosage regimen.

In some embodiments, the pharmaceutical compositions of the present disclosure are adapted for oral administration. As used herein, the term “oral administration” refers to administration of a compound of the present disclosure to the mouth of a patient for ingestion into the gastrointestinal tract. In some embodiments, the pharmaceutical compositions of the present disclosure can be formulated into conventional oral dosage forms including without limitation capsules, tablets, powders, and liquid preparations such as suspensions, solutions, elixirs, syrups, concentrated drops, and the like. In some embodiments, a compound of the present disclosure may be combined with solid excipients, optionally grinding a resulting mixture, and optionally processing the mixture of granules, after adding suitable auxiliaries, if desired, to obtain, for example, tablets, coated tablets, hard capsules, soft capsules, solutions (e.g., aqueous, alcoholic or oily solutions), and the like. In some embodiments, excipients suitable for use in the oral pharmaceutical compositions of the present disclosure include without limitation fillers such as sugars, including lactose, glucose, sucrose, mannitol, or sorbitol; cellulose preparations, for example, corn starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP or povidone); and oily excipients, including vegetable and animal oils, such as sunflower oil, olive oil, or cod liver oil. In some embodiments, the oral pharmaceutical compositions of the present disclosure may also contain disintegrating agents, such as, for example, cross-linked polyvinylpyrrolidone, agar, or alginic acid, or a salt thereof such as sodium alginate; a lubricant, such as talc or magnesium stearate; a plasticizer, such as glycerol or sorbitol; a sweetening agent such as sucrose, fructose, lactose, or aspartame; a natural or artificial flavoring agent, such as, for example, peppermint, oil of wintergreen, or cherry flavoring; or dye-stuffs or pigments, which may be used for identification or characterization of different doses or combinations. In some embodiments, the oral pharmaceutical compositions of the present disclosure may also contain dragée cores with suitable coatings. In some embodiments, concentrated sugar solutions may be used, which may optionally contain, for example, gum arabic, talc, poly-vinylpyrrolidone, carbopol gel, polyethylene glycol, titanium dioxide, lacquer solutions, and suitable organic solvents or solvent mixtures.

In some embodiments, the pharmaceutical compositions of the present disclosure that can be used orally include without limitation push-fit capsules made of gelatin (“gelcaps”), as well as soft, sealed capsules made of gelatin, and a plasticizer, such as glycerol or sorbitol. In some embodiments, the push-fit capsules can contain a compound of the present disclosure in admixture with filler such as lactose, binders such as starches, and/or lubricants such as talc or magnesium stearate and, optionally, stabilizers. In some embodiments including soft capsules, the active compound may be dissolved or suspended in suitable liquids, such as fatty oils, liquid paraffin, liquid polyethylene glycols, and the like.

In some embodiments, the pharmaceutical compositions of the present disclosure are adapted for inhalation administration. As used herein, the term “inhalation administration” refers to delivery of a compound of the present disclosure by passage through a patient's nose or mouth during inhalation and passage of the compound through the walls of the lungs of the patient. In some embodiments, the pharmaceutical compositions of the present disclosure suitable for inhalation administration may be formulated as dry powder or a suitable solution, suspension or aerosol. In some embodiments, powders and solutions may be formulated with suitable additives known in the art. In some embodiments, powders may include a suitable powder base such as lactose or starch. In some embodiments, solutions may comprise propylene glycol, sterile water, ethanol, sodium chloride, and other additives, such as, for example, acid, alkali, and buffer salts. In some embodiments, such solutions or suspensions may be administered by inhaling via a spray, pump, atomizer, nebulizer, and the like. In some embodiments, the pharmaceutical compositions of the present disclosure suitable for inhalation administration may also be used in combination with other inhaled therapies, including without limitation corticosteroids such as, for example, fluticasone proprionate, beclomethasone dipropionate, triamcinolone acetonide, budesonide, and mometasone furoate; beta agonists such as, for example, albuterol, salmeterol, and formoterol; anticholinergic agents such as, for example, ipratroprium bromide or tiotropium; vasodilators such as, for example, treprostinal and iloprost; enzymes such as, for example, DNAase; therapeutic proteins; immunoglobulin antibodies; an oligonucleotide, such as, for example, single or double stranded DNA or RNA, siRNA; antibiotics such as, for example, tobramycin; muscarinic receptor antagonists; leukotriene antagonists; cytokine antagonists; protease inhibitors; cromolyn sodium; nedocril sodium; and sodium cromoglycate.

In some embodiments, the pharmaceutical compositions of the present disclosure are adapted for intravesical administration. As used herein, the term “intravesical administration” refers to delivery of a compound of the present disclosure directly into the bladder of a patient. In some embodiments, the pharmaceutical composition is administered via a catheter. In some embodiments, the catheter is a urethral catheter.

In some embodiments, the pharmaceutical compositions of the present disclosure are adapted for parenteral administration. As used herein, the term “parenteral administration” refers to a compound of the present disclosure being injected or infused into a patient and includes without limitation intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the pharmaceutical compositions of the present disclosure suitable for parenteral administration may be formulated in sterile liquid solutions, including without limitation physiologically compatible buffers or solutions, such as, for example, saline solution, Hank's solution or Ringer's solution. In some embodiments, the pharmaceutical compositions of the present disclosure suitable for parenteral administration may be prepared as dispersions in non-aqueous solutions, such as, for example, glycerol, propylene glycol, ethanol, liquid polyethylene glycols, triacetin, vegetable oils, and the like. In some embodiments, solutions may also contain a preservative, such as, for example, methylparaben, propylparaben, chlorobutanol, phenol, sorbic acid, thimerosal, and the like. In addition, pharmaceutical compositions of the present disclosure suitable for parenteral administration may be formulated in solid form, including, for example, lyophilized forms, and redissolved or suspended prior to use. In some embodiments, the pharmaceutical composition is administered via a needle.

In some embodiments, the present disclosure provides methods and compositions of pretreating a catheter with a compound of the present disclosure, for example, to prevent an infection after the catheter is inserted into a patient. In some embodiments, a method of the present disclosure includes coating a catheter with a compound of the present disclosure prior to inserting the catheter into a patient. In some embodiments, the present disclosure provides a composition comprising a catheter coated with a compound of the present disclosure. In some embodiments, such methods and compositions may be used as a prophylactic treatment of an infection in a patient.

The present disclosure also provides methods of treatment. As used herein, the terms “treating,” “treatment,” “therapy,” and like terms refer to administration of a compound or pharmaceutical composition of the present disclosure in an amount effective to prevent, alleviate or ameliorate one or more symptoms of a disease or condition (i.e., indication) and/or to prolong the survival of the patient being treated. In some embodiments, “treating,” “treatment,” “therapy,” and like terms also include without limitation reducing or eliminating infection in a patient.

In carrying out the methods of the present disclosure, an effective amount of a compound of the present disclosure is administered to a patient in need thereof. As used herein, the term “effective amount,” in the context of administration, refers to the amount of a compound or pharmaceutical composition of the present disclosure that when administered to a patient is sufficient to prevent, alleviate or ameliorate one or more symptoms of a disease or condition (i.e., indication) and/or to prolong the survival of the patient being treated. Such an amount should result in no or few adverse events in the treated patient. Similarly, such an amount should result in no or few toxic effects in the treated patient. As those familiar with the art will understand, the amount of a compound or pharmaceutical composition of the present disclosure will vary depending upon a number of factors, including without limitation the activity of a compound of the present disclosure (in vitro, e.g. a compound of the present disclosure vs. target, or in vivo activity in animal efficacy models), pharmacokinetic results in animal models (e.g., biological half-life or bioavailability), the type of patient being treated, the patient's age, size, weight, and general physical condition, the disorder associated with the patient, and the dosing regimen being employed in the treatment.

In some embodiments of the present disclosure, an effective amount of a compound of the present disclosure to be delivered to a patient in need thereof can be quantified by determining micrograms of a compound of the present disclosure per kilogram of patient body weight. In some embodiments, the amount of a compound of the present disclosure administered to a patient is from about 0.1 to about 1000 milligram (mg) of a compound of the present disclosure per kilogram (kg) of patient body weight. In some embodiments, the amount of a compound of the present disclosure administered to a patient is from about 0.1 to about 500 mg of a compound of the present disclosure per kg of patient body weight. In some embodiments, the amount of a compound of the present disclosure administered to a patient is from about 0.1 to about 300 mg of a compound of the present disclosure per kg of patient body weight. In some embodiments, the amount of a compound of the present disclosure administered to a patient is from about 0.1 to about 200 mg of a compound of the present disclosure per kg of patient body weight. In some embodiments, the amount of a compound of the present disclosure administered to a patient is from about 0.1 to about 100 mg of a compound of the present disclosure per kg of patient body weight. As those of ordinary skill in the art understand multiple doses may be used.

In some embodiments of the present disclosure, a compound of the present disclosure is administered as a multiple dose regimen. As used herein, the term “multiple dose regimen” refers to a treatment time period of more than one day. In some embodiments of the present disclosure, the multiple dose regimen is a time period of up to about 2 days. In some embodiments of the present disclosure, the multiple dose regimen is a time period of up to about 3 days. In some embodiments of the present disclosure, the multiple dose regimen is a time period of up to about 4 days. In some embodiments of the present disclosure, the multiple dose regimen is a time period of up to about 5 days. In some embodiments of the present disclosure, the multiple dose regimen is a time period of up to about 6 days. In some embodiments of the present disclosure, the multiple dose regimen is a time period of up to about 7 days. In some embodiments of the present disclosure, the multiple dose regimen is a time period of up to about 14 days. In some embodiments of the present disclosure, the multiple dose regimen is a time period of up to about one month. In some embodiments of the present disclosure, the multiple dose regimen is a time period of up to about two months. In some embodiments of the present disclosure, the multiple dose regimen is a time period of up to about three months. In some embodiments of the present disclosure, the multiple dose regimen is a time period of up to about four months. In some embodiments of the present disclosure, the multiple dose regimen is a time period of up to about five months. In some embodiments of the present disclosure, the multiple dose regimen is a time period of up to about six months. Other time periods may be used herein.

In some embodiments of the present disclosure, a compound of the present disclosure is administered as part of a chronic treatment regimen. As used herein, the term “chronic treatment regimen” refers to treatment with a compound of the present disclosure over an extended period of time during a patient's lifetime. In some embodiments, chronic treatment is lifelong treatment.

In some embodiments of the present disclosure, a compound of the present disclosure is administered as a single dose. In some embodiments of the present disclosure, a compound of the present disclosure is administered as a single unit dose. As used herein, the term “unit dose” is a predetermined amount of a compound of the present disclosure. The amount of a compound of the present disclosure is generally equal to the dosage of a compound of the present disclosure that would be administered to a patient or a convenient fraction of such a dosage such as, for example, one-half or one-third of such a dosage. According to the methods of the present disclosure, the terms “single dose” and “single unit dose” include embodiments wherein the composition can be administered as a single application and administered as multiple applications.

In some embodiments, a compound of the present disclosure may also be used in combination with one or more additional active ingredients for treating the same disease or condition. In some embodiments, such combination use includes administration of a compound of the present disclosure and one or more additional active ingredient at different times, or co-administration of a compound of the present disclosure and one or more additional active ingredients. In some embodiments, dosage may be modified for a compound of the present disclosure or one or more additional active ingredients used in combination, e.g., reduction in the amount dosed relative to a compound of the present disclosure or one or more additional active ingredients used alone, by methods well known to those of ordinary skill in the art. In some embodiments, co-administration includes simultaneous administration of a compound of the present disclosure and an additional active ingredient in the same dosage form, simultaneous administration of a compound of the present disclosure and an additional active ingredient in separate dosage forms, and separate administration of a compound of the present disclosure and an additional active ingredient.

It is understood that use in combination includes use with one or more additional active ingredients or other medical procedure in which the one or more additional active ingredients or other medical procedure may be administered at different times (e.g., within a short time, such as within hours (e.g., 1, 2, 3, 4-24 hours, etc.), or within a longer time (e.g. 1-2 days, 2-4 days, 4-7 days, 1-4 weeks, etc.)) than a compound or pharmaceutical composition of the present disclosure, or at the same time as a compound or pharmaceutical composition of the present disclosure. Use in combination also includes use with one or more additional active ingredients or other medical procedure that is administered once or infrequently, such as surgery, along with a compound or pharmaceutical composition of the present disclosure administered within a short time or longer time before or after the administration of the one or more additional active ingredients or completion of the other medical procedure.

In some embodiments, the present disclosure provides for delivery of a compound or pharmaceutical composition of the present disclosure and one or more additional active ingredients delivered by a different route of administration or by the same route of administration. In some embodiments, the use in combination for any route of administration includes delivery of a compound or pharmaceutical composition of the present disclosure and one or more additional active ingredients delivered by the same route of administration together in any pharmaceutical composition, including pharmaceutical compositions in which the two compounds are chemically linked in such a way that such compounds maintain their therapeutic activity when administered.

In some embodiments, the one or more additional active ingredients may be co-administered with a compound or pharmaceutical composition of the present disclosure. In some embodiments, use in combination by co-administration includes administration of co-formulations or formulations of chemically joined compounds, or administration of two or more compounds in separate formulations within a short time of each other (e.g., within an hour, 2 hours, 3 hours, up to 24 hours, etc.), administered by the same or different routes. In some embodiments, co-administration of separate formulations includes co-administration by delivery via one device, for example, the same inhalant device, the same syringe, etc., or administration from separate devices within a short time of each other. In some embodiments, co-formulations of a compound or pharmaceutical composition of the present disclosure and one or more additional active ingredients delivered by the same route includes preparation of the materials together such that they can be administered by one device, including the separate compounds combined in one formulation, or compounds that are modified such that the compounds are chemically joined, yet still maintain their biological activity. In some embodiments, such chemically joined compounds may have a linkage that is substantially maintained in vivo, or the linkage may break down in vivo, separating the two active components.

The present disclosure also provides a method of treating an infection in a patient in need thereof. In some embodiments, the method comprises administering an effective amount of a compound of the present disclosure, or a pharmaceutically acceptable salt thereof, to the patient.

As used herein, the term “infection” refers to any microbe infection of a patient's body. Infection includes the invasion of a patient's body by a microbe and subsequent multiplication in the patient's body.

The present disclosure also provides a method of treating an infection of a lower extremity ulcer in a patient in need thereof. In some embodiments, the method comprises administering an effective amount of a compound of the present disclosure, or a pharmaceutically acceptable salt thereof, to the patient. As used herein, the term “infection” refers to any microbe infection of a patient's body. Infection includes the invasion of a patient's body by a microbe and subsequent multiplication in the patient's body. As used herein, the term “lower extremity” refers to a lower limb of a patient's body, including without limitation the hip, thigh, leg, ankle, and foot. As used herein, the term “ulcer” refers to an open wound found anywhere on the lower extremity of a patient.

In some embodiments, the present disclosure provides a method of treating an infection of a diabetic foot ulcer in a patient in need thereof. In some embodiments, the method comprises administering an effective amount of a compound of the present disclosure, or a pharmaceutically acceptable salt thereof, to the patient. In some embodiments, the patient is suffering from Type I diabetes or Type II diabetes. As used herein, the term “diabetic foot ulcer” refers to an open wound located anywhere on the foot of a patient. In some embodiments, the wound is located on the heel, mid-foot, and/or forefoot of the patient's foot. As used herein, the term “treating,” in the context of a diabetic foot ulcer, also includes without limitation reducing or eliminating infection in a patient, which, in some embodiments, results in limiting the progression in size, area, and/or depth of the foot ulcer; reducing the size, area, and/or depth of the foot ulcer; increasing the rate of healing and/or reducing time to healing; healing of the foot ulcer (about 100% epithelialization with no drainage); and/or decreased incidence of amputation or slowing in time to amputation.

In some embodiments, the patient is a human. In some embodiments, the administration is topical administration. In some embodiments, the administration is carried out using the compound, or a pharmaceutically acceptable salt thereof, in a lotion, paste, gel, cream, ointment, oil or other viscous composition. In some embodiments, the patient is administered at least one additional active ingredient. In some embodiments, the administration is carried out as a multiple dose regimen. In some embodiments, the multiple dose regimen is a time period of up to about one month. In some embodiments of the present disclosure, the multiple dose regimen is a time period of up to about two months. In some embodiments of the present disclosure, the multiple dose regimen is a time period of up to about three months. In some embodiments of the present disclosure, the multiple dose regimen is a time period of up to about four months. Other time periods may be used herein.

In some embodiments, the administration is carried out one or more times per day. In some embodiments, the administration is carried out one time per day. In some embodiments, the administration is carried out two times per day. In some embodiments, the administration is carried out three times per day. In some embodiments, the administration is carried out four times per day.

The present disclosure also provides a method of treating a urinary tract infection in a patient in need thereof. In some embodiments, the method comprises administering an effective amount of a compound of the present disclosure, or a pharmaceutically acceptable salt thereof, to the patient. As used herein, the term “urinary tract” refers to the organs of a patient's body that produce, store, and discharge urine and includes without limitation the kidneys, ureters, bladder, and urethra. As used herein, the term “urinary tract infection” refers to an infection of the urinary tract of a patient and includes without limitation an uncomplicated urinary tract infection and a complicated urinary tract infection. As used herein, the term “uncomplicated urinary tract infection” refers to an infection by a microbe of a structurally and functionally normal urinary tract of a patient. As used herein, the term “complicated urinary tract infection” refers to an infection by a microbe of an abnormal structural and functional urinary tract of a patient. In some embodiments, the complicated urinary tract infection is a catheter-associated urinary tract infection. As used herein, the term “catheter-associated urinary tract infection” refers to a complicated urinary tract infection that occurs in a patient having an indwelling urinary catheter.

In some embodiments, the patient is a human. In some embodiments, the administration is intravesical administration. In some embodiments, the administration is carried out using a compound of the present disclosure, or a pharmaceutically acceptable salt thereof, in a liquid solution or suspension. In some embodiments, the patient is administered at least one additional active ingredient. In some embodiments, the administration is carried out as a multiple dose regimen. In some embodiments of the present disclosure, the multiple dose regimen is a time period of up to about 2 days. In some embodiments of the present disclosure, the multiple dose regimen is a time period of up to about 3 days. In some embodiments of the present disclosure, the multiple dose regimen is a time period of up to about 4 days. In some embodiments of the present disclosure, the multiple dose regimen is a time period of up to about 5 days. In some embodiments of the present disclosure, the multiple dose regimen is a time period of up to about 6 days. In some embodiments of the present disclosure, the multiple dose regimen is a time period of up to about 7 days. In some embodiments of the present disclosure, the multiple dose regimen is a time period of up to about 14 days. In some embodiments of the present disclosure, the administration is carried out as a chronic treatment regimen. Other time periods may be used herein.

In some embodiments, the administration is carried out one or more times per day. In some embodiments, the administration is carried out one time per day. In some embodiments, the administration is carried out two times per day. In some embodiments, the administration is carried out three times per day. In some embodiments, the administration is carried out four times per day.

The present disclosure also provides a method of treating a lung infection in a patient in need thereof. In some embodiments, the method comprises administering an effective amount of a compound of the present disclosure, or a pharmaceutically acceptable salt thereof, to the patient.

As used herein, the term “lung infection” refers to an infection of one or both of a patient's lungs.

In some embodiments, the lung infection arises from a pulmonary condition. As used herein, the term “pulmonary condition” refers to both infection and non-infection induced disease and dysfunction of the respiratory system.

Non-limiting examples of pulmonary conditions include without limitation genetic conditions, acquired conditions, primary conditions, secondary conditions, asthma, chronic obstructive pulmonary disease, cystic fibrosis, bronchiolitis, pneumonia, bronchitis, emphysema, adult respiratory distress syndrome, allergies, lung cancer, small cell lung cancer, primary lung cancer, metastatic lung cancer, bronchiestasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, and conditions caused by any one or combination of the above.

In some embodiments, the pulmonary condition is caused by a coronavirus. In some embodiments, the coronavirus is selected from the group consisting of an alphacoronavirus, a betacoronavirus, a gammacoronavirus, a deltacoronavirus, and an omicroncoronavirus, or combinations thereof.

In some embodiments, the coronavirus is selected from the group consisting of porcine epidemic diarrhea virus (PEDv), scotophilus bat coronavirus 512, bat coronavirus CDPHE15, BtRF-Alpha-CoV HuB-2013, bat coronavirus HKU10, miniopterus bat coronavirus HKU8, miniopterus bat coronavirus 1, Nyctalus velutinus alphacoronavirus SC-2013, Pipistrellus kuhlii coronavirus 3398, Myotis ricketti alphacoronavirus Sax-2011, HumCoV 229E, 229E-related bat coronavirus, camel alphacoronavirus, alpaca respiratory coronavirus, HumCoV NL63, NL63-related Bat-CoV BtKYNL63-9b, HKU2, SADSr-CoV, SADS-CoV, Lucheng Rn rat coronavirus2, FIPV, TGEV, PRCV, alphacoronavirus 1, mink coronavirus 1, FRCoV-NL-2010, Sorex araneus coronavirus T14, Suncus murinus coronavirus X74, HumCoV OC43, HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYNO2, human SARS-CoV-2, cat-CoV, tiger-CoV, dog-CoV, mink-CoV, GD pangolin-CoV, bat Hp-betacoronavirus Zhejiang2013, peafowl IBV, avian coronavirus 9203, fowl IBV, avian coronavirus, duck-CoV, partridge IBV, goose coronavirus CB17, beluga whale coronavirus SW1, HKU22, HKU19, HKU20, HKU21, HKU11, HKU12, HKU16, HKU27, HKU28, HKU29, HKU18, HKU13, HKU30-UAE, HKU30-Poland, HKU17-USA, HKU17-China, AlCCoV, and HKU15, or combinations thereof.

In some embodiments, the coronavirus is a betacoronavirus selected from the group consisting of HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYNO2, human SARS-CoV-2, cat-CoV, tiger-CoV, dog-CoV, mink-CoV, GD pangolin-CoV, and bat Hp-betacoronavirus Zhejiang2013, or combinations thereof.

In some embodiments of the present disclosure, a method of treating a lung infection arising from cystic fibrosis in a patient in need thereof is provided. In some embodiments, the method comprises administering an effective amount of a compound of the present disclosure, or a pharmaceutically acceptable salt thereof, to the patient. As used herein, the term “cystic fibrosis” refers to a genetic disease that causes the production of abnormally thick mucus resulting in lung infections and damage to the lungs, digestive system, and other organs in a patient's body.

In some embodiments, the administration is inhalation administration. In some embodiments, the patient is administered at least one additional active ingredient. In some embodiments, the patient is a human. In some embodiments, the administration is carried out using a compound of the present disclosure, or a pharmaceutically acceptable salt thereof, in a liquid solution, suspension or dry powder. In some embodiments, the administration is carried out as a multiple dose regimen. In some embodiments, the multiple dose regimen is a time period of up to about one month. In some embodiments of the present disclosure, the multiple dose regimen is a time period of up to about two months. In some embodiments of the present disclosure, the multiple dose regimen is a time period of up to about three months. In some embodiments of the present disclosure, the multiple dose regimen is a time period of up to about four months. In some embodiments of the present disclosure, the multiple dose regimen is a time period of up to about five months. In some embodiments of the present disclosure, the multiple dose regimen is a time period of up to about six months. In some embodiments of the present disclosure, the multiple dose regimen is a time period of up to about seven months. In some embodiments of the present disclosure, the multiple dose regimen is a time period of up to about eight months. Other time periods may be used herein.

In some embodiments, the administration is carried out one or more times per day. In some embodiments, the administration is carried out one time per day. In some embodiments, the administration is carried out two times per day. In some embodiments, the administration is carried out three times per day. In some embodiments, the administration is carried out four times per day.

In some embodiments of the present disclosure, a method of treating pneumonia in a patient in need thereof is provided. In some embodiments, the method comprises administering an effective amount of a compound of the present disclosure, or a pharmaceutically acceptable salt thereof, to the patient. As used herein, the term “pneumonia” refers to an infection by a microbe of one or both lungs of a patient resulting in inflammation of lung tissue. In some embodiments, the pneumonia is ventilator acquired pneumonia. As used herein, the term “ventilator acquired pneumonia” refers to pneumonia arising from a patient being connected to a mechanical ventilation machine. Ventilator acquired pneumonia includes pneumonia occurring more than about 48 hours after a patient has been intubated and received mechanical ventilation.

In some embodiments, the administration is inhalation administration. In some embodiments, the patient is administered at least one additional active ingredient. In some embodiments, the patient is a human. In some embodiments, the administration is carried out using a compound of the present disclosure, or a pharmaceutically acceptable salt thereof, in a liquid solution, suspension or dry powder. In some embodiments, the administration is carried out as a multiple dose regimen. In some embodiments of the present disclosure, the multiple dose regimen is a time period of up to about 7 days. In some embodiments of the present disclosure, the multiple dose regimen is a time period of up to about 14 days. In some embodiments of the present disclosure, the multiple dose regimen is a time period of up to about 21 days. In some embodiments, the multiple dose regimen is a time period of up to about one month. In some embodiments of the present disclosure, the multiple dose regimen is a time period of up to about two months. Other time periods may be used herein.

In some embodiments, the administration is carried out one or more times per day. In some embodiments, the administration is carried out one time per day. In some embodiments, the administration is carried out two times per day. In some embodiments, the administration is carried out three times per day. In some embodiments, the administration is carried out four times per day.

The present disclosure also provides a method of treating an infection in a burn wound in a patient in need thereof. In some embodiments, the method comprises administering an effective amount of a compound of the present disclosure, or a pharmaceutically acceptable salt thereof, to the patient. As used herein, the term “burn wound” refers to a burn injury to a patient's body involving damage to a patient's skin and possibly tissues underlying the patient's skin. There are three primary types of burn levels known to one of skill in the art, including without limitation first-, second-, and third-degree burns. In some embodiments, the method of treating an infection in a burn wound contemplated by the present disclosure is used to treat a first-, second-, and/or third-degree burn.

In some embodiments, the patient is a human. In some embodiments, the administration is topical administration. In some embodiments, the administration is carried out using a compound of the present disclosure, or a pharmaceutically acceptable salt thereof, in lotion, cream, ointment, oil, solution, suspension, emulsion or other viscous composition. In some embodiments, the patient is administered at least one additional active ingredient. In some embodiments, the administration is carried out as a multiple dose regimen. In some embodiments of the present disclosure, the multiple dose regimen is a time period of up to about 2 days. In some embodiments of the present disclosure, the multiple dose regimen is a time period of up to about 3 days. In some embodiments of the present disclosure, the multiple dose regimen is a time period of up to about 4 days. In some embodiments of the present disclosure, the multiple dose regimen is a time period of up to about 5 days. In some embodiments of the present disclosure, the multiple dose regimen is a time period of up to about 6 days. In some embodiments of the present disclosure, the multiple dose regimen is a time period of up to about 7 days. In some embodiments of the present disclosure, the multiple dose regimen is a time period of up to about 14 days. In some embodiments of the present disclosure, the multiple dose regimen is a time period of up to about 21 days. In some embodiments, the multiple dose regimen is a time period of up to about one month. In some embodiments, the multiple dose regimen is a time period of up to about two months. Other time periods may be used herein.

In some embodiments, the administration is carried out one or more times per day. In some embodiments, the administration is carried out one time per day. In some embodiments, the administration is carried out two times per day. In some embodiments, the administration is carried out three times per day. In some embodiments, the administration is carried out four times per day.

The present disclosure also provides a method of treating otitis externa in a patient in need thereof. In some embodiments, the method comprises administering an effective amount of a compound of the present disclosure, or a pharmaceutically acceptable salt thereof, to the patient.

As used herein, the term “otitis externa” refers to an infection of the external ear canal of a patient.

In some embodiments, the patient is a human. In some embodiments, the administration is topical administration directly into the patient's external ear canal. In some embodiments, the administration is carried out using a compound of the present disclosure, or a pharmaceutically acceptable salt thereof, in a liquid solution, suspension, lotion, paste, gel, cream, ointment, oil or other viscous composition. In some embodiments, the patient is administered at least one additional active ingredient. In some embodiments, the administration is carried out as a multiple dose regimen. In some embodiments of the present disclosure, the multiple dose regimen is a time period of up to about 7 days. In some embodiments of the present disclosure, the multiple dose regimen is a time period of up to about 14 days. In some embodiments of the present disclosure, the multiple dose regimen is a time period of up to about 21 days. In some embodiments, the multiple dose regimen is a time period of up to about one month. Other time periods may be used herein.

In some embodiments, the administration is carried out one or more times per day. In some embodiments, the administration is carried out one time per day. In some embodiments, the administration is carried out two times per day. In some embodiments, the administration is carried out three times per day. In some embodiments, the administration is carried out four times per day.

The present disclosure also provides a method of treating bacterial vaginosis in a patient in need thereof. In some embodiments, the method comprises administering an effective amount of a compound of the present disclosure, or a pharmaceutically acceptable salt thereof, to the patient.

As used herein, the term “bacterial vaginosis” refers to an infection of the vagina of a patient caused by an overgrowth of bacteria naturally found in the vagina.

In some embodiments, the patient is a female human. In some embodiments, the administration is topical administration. In some embodiments, the administration is carried out using a compound of the present disclosure, or a pharmaceutically acceptable salt thereof, in a lotion, gel, cream, ointment, oil, solution, suspension, emulsion or other viscous composition. In some embodiments, the patient is administered at least one additional active ingredient. In some embodiments, the administration is carried out as a multiple dose regimen. In some embodiments of the present disclosure, the multiple dose regimen is a time period of up to about 7 days. In some embodiments of the present disclosure, the multiple dose regimen is a time period of up to about 14 days. In some embodiments of the present disclosure, the multiple dose regimen is a time period of up to about 21 days. In some embodiments, the multiple dose regimen is a time period of up to about one month. Other time periods may be used herein.

In some embodiments, the administration is carried out one or more times per day. In some embodiments, the administration is carried out one time per day. In some embodiments, the administration is carried out two times per day. In some embodiments, the administration is carried out three times per day. In some embodiments, the administration is carried out four times per day.

The present disclosure also provides a method of treating impetigo in a patient in need thereof. In some embodiments, the method comprises administering an effective amount of a compound of the present disclosure, or a pharmaceutically acceptable salt thereof, to the patient.

As used herein, the term “impetigo” refers an infection of the skin of a patient that results in vesicles, pustules, yellowish crusts, and the like.

In some embodiments, the patient is a human. In some embodiments, the administration is topical administration. In some embodiments, the administration is carried out using a compound of the present disclosure, or a pharmaceutically acceptable salt thereof, in a lotion, gel, cream, ointment, oil, solution, suspension, emulsion or other viscous composition. In some embodiments, the patient is administered at least one additional active ingredient. In some embodiments, the administration is carried out as a multiple dose regimen. In some embodiments of the present disclosure, the multiple dose regimen is a time period ofup to about 2 days. In some embodiments of the present disclosure, the multiple dose regimen is a time period of up to about 3 days. In some embodiments of the present disclosure, the multiple dose regimen is a time period of up to about 4 days. In some embodiments of the present disclosure, the multiple dose regimen is a time period of up to about 5 days. In some embodiments of the present disclosure, the multiple dose regimen is a time period of up to about 6 days. In some embodiments of the present disclosure, the multiple dose regimen is a time period of up to about 7 days. In some embodiments of the present disclosure, the multiple dose regimen is a time period of up to about 14 days. In some embodiments of the present disclosure, the multiple dose regimen is a time period of up to about 21 days. In some embodiments, the multiple dose regimen is a time period of up to about one month. Other time periods may be used herein.

In some embodiments, the administration is carried out one or more times per day. In some embodiments, the administration is carried out one time per day. In some embodiments, the administration is carried out two times per day. In some embodiments, the administration is carried out three times per day. In some embodiments, the administration is carried out four times per day.

The present disclosure also provides a method of treating oral mucositis in a patient in need thereof. In some embodiments, the method comprises administering an effective amount of a compound of the present disclosure, or a pharmaceutically acceptable salt thereof, to the patient.

As used herein, the term “oral mucositis” refers to inflammation and ulceration in the mouth. Oral mucositis is a common complication experienced by patients receiving cancer chemotherapy or radiation treatment. Oral mucositis can lead to several problems, including pain, nutritional problems as a result of inability to eat, and increased risk of infection due to open sores in the mucosa. Oral mucositis may also have a significant effect on a cancer patient's quality of life and can limit the effectiveness of certain treatment options (i.e., requiring a reduction in subsequent chemotherapy doses). Oral mucositis is also a significant side effect of bone marrow transplantation.

In some embodiments, the patient is a human. In some embodiments, the administration is topical administration directly into the patient's oral cavity. In some embodiments, the administration is carried out using a compound of the present disclosure, or a pharmaceutically acceptable salt thereof, in a liquid solution, suspension, lotion, paste, gel, cream, ointment, oil or other viscous composition. In some embodiments, the administration is carried out using the compound of the present disclosure in a mouthwash. In some embodiments, the compound of the present disclosure is present in the pharmaceutical composition in an amount from about 1% to about 20% (weight/weight). In some embodiments, the compound of the present disclosure is present in the pharmaceutical composition in an amount from about 5% to about 15% (weight/weight). In some embodiments, the compound of the present disclosure is present in the pharmaceutical composition in an amount from about 30% to about 50% (weight/weight).

In some embodiments, the patient is administered at least one additional active ingredient.

In some embodiments, the administration is carried out as a multiple dose regimen. In some embodiments of the present disclosure, the multiple dose regimen is a time period of up to about 7 days. In some embodiments of the present disclosure, the multiple dose regimen is a time period of up to about 14 days. In some embodiments of the present disclosure, the multiple dose regimen is a time period ofup to about 21 days. In some embodiments, the multiple dose regimen is a time period of up to about one month. Other time periods may be used herein.

In some embodiments, the administration is carried out one or more times per day. In some embodiments, the administration is carried out one time per day. In some embodiments, the administration is carried out two times per day. In some embodiments, the administration is carried out three times per day. In some embodiments, the administration is carried out four times per day.

The present disclosure also provides a kit. In some embodiments, the kit comprises a compound according to the present disclosure, or a pharmaceutically acceptable salt thereof, or a pharmaceutical composition according to the present disclosure. As used herein, the term “kit” refers to any manufacture, such as, for example, a package, container, and the like, containing a compound according to the present disclosure, or a pharmaceutically acceptable salt thereof, or a pharmaceutical composition according to the present disclosure. In some embodiments, a compound according to the present disclosure, or a pharmaceutically acceptable salt thereof, or a pharmaceutical composition according to the present disclosure is packaged in a vial, bottle, tube, flask or patch, which may be further packaged within a box, envelope, bag, or the like. In some embodiments, a compound according to the present disclosure, or a pharmaceutically acceptable salt thereof, or a pharmaceutical composition according to the present disclosure is approved by the U.S. Food and Drug Administration or similar regulatory agency in the U.S. or a jurisdiction or territory outside the U.S. for administration to a patient. In some embodiments, the kit includes written instructions for use and/or other indication that a compound according to the present disclosure, or a pharmaceutically acceptable salt thereof, or a pharmaceutical composition according to the present disclosure is suitable or approved for administration to a patient. In some embodiments, a compound or composition of the present disclosure is packaged in unit dose or single unit dose form, such as, for example, single unit dose pills, capsules or the like. In some embodiments, the kit includes a dispenser.

The present disclosure also provides the use of a compound of the present disclosure, or a pharmaceutically acceptable salt thereof, for the manufacture of a medicament. As used herein, the term “medicament” refers to a pharmaceutical composition according to the present disclosure.

In some embodiments, the pharmaceutical composition is contained in any manufacture, such as, for example, a package, container, and the like.

In addition to the aspects and embodiments described and provided elsewhere in the present disclosure, the following non-limiting list of embodiments are also contemplated.
1. A compound having the formula:

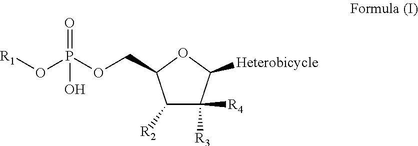

or a pharmaceutically acceptable salt thereof, wherein
R₁ is (C₁-C₈)straight-chain alkyl, (C₃-C₈)branched-chain alkyl, (C₃-C₈)cycloalkyl, phenylalkyl, alkylcycloalkyl, hydroxy(C₁-C₈)straight-chain alkyl, hydroxy(C₃-C₈)branched-chain alkyl, alkoxy(C₁-C₈)straight-chain alkyl, alkoxy(C₃-C₈)branched-chain alkyl, halo(C₁-C₈)straight-chain alkyl, or halo(C₃-C₈)branched-chain alkyl; R₂, R₃, and R₄ are each independently H, OH, oxy(C₁-C₈)straight-chain alkyl, oxy(C₁-C₈)branched-chain alkyl, CF₃, or F;
Heterobicycle is selected from the group consisting of

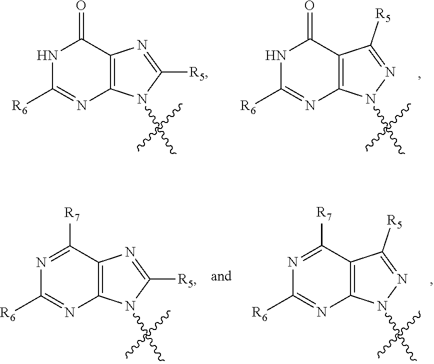

R₅ is H, (C₁-C₅)straight-chain alkyl, (C₁-C₅)branched-chain alkyl, or CF₃;
R₆ and R₇ are each independently selected from H, N(R₈)R₉, (C₁-C₅)straight-chain alkyl, (C₁-C₅)branched-chain alkyl, or CF₃; and
R₈ and R₉ are each independently selected from H, (C₁-C₅)straight-chain alkyl, (C₁-C₅)branched-chain alkyl, or are bound to one another through a C—C bond to form a C₃-C₈ cycloalkyl.
2. The compound according to clause 1, wherein R₁ is octyl.
3. The compound according to clause 1, wherein R₁ is hexyl.
4. The compound according to clause 1, wherein R₁ is butyl.
5. The compound according to clause 1, wherein R₁ is 2-methylpropyl.
6. The compound according to clause 1, wherein R₁ is 2-methylbutyl.
7. The compound according to clause 1, wherein R₁ is 1-butanolyl.
8. The compound according to any one of clauses 1 to 7, wherein R₂ is H.
9. The compound according to any one of clauses 1 to 7, wherein R₂ is OH.
10. The compound according to any one of clauses 1 to 9, wherein R₃ and R₄ are both H.
11. The compound according to any one of clauses 1 to 9, wherein R₃ and R₄ are both F.
12. The compound according to any one of clauses 1 to 9, wherein R₃ is OCH₃ and R₄ is H.
13. The compound according to any one of clauses 1 to 9, wherein R₃ is H and R₄ is OCH₃.
14. The compound according to any one of clauses 1 to 13, wherein Heterobicycle is

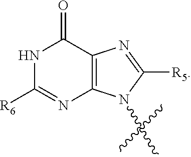

15. The compound according to clause 14, wherein R₅ is H and R₆ is NH₂.
16. The compound according to clause 15, wherein R₃ is OCH₃.
17. The compound according to any one of clauses 1 to 13, wherein Heterobicycle is

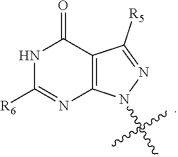

18. The compound according to any one of clauses 1 to 13, wherein Heterobicycle is

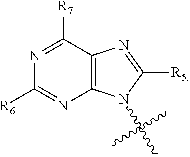

19. The compound according to clause 18, wherein R₅ and R₆ are both H and R₇ is NH₂.
20. The compound according to clause 18, wherein R₅ and R₇ are both H and R₆ is NH₂.
21. The compound according to any one of clauses 1 to 13, wherein Heterobicycle is

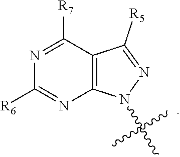

22. The compound according to clause 21, wherein R₅ and R₆ are both H and R₇ is NH₂.
23. A compound having the formula:

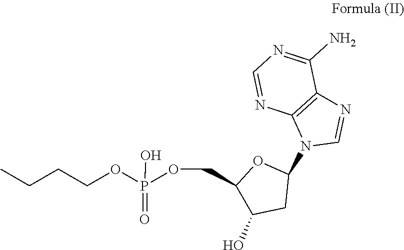 [SMILES: CCCCOP(=O)(O)OC[C@H]1O[C@@H](n2cnc3c(N)ncnc32)C[C@@H]1O]

or a pharmaceutically acceptable salt thereof.
24. A compound having the formula:

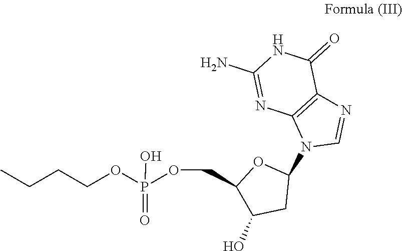 [SMILES: CCCCOP(=O)(O)OC[C@H]1O[C@@H](n2cnc3c(=O)[nH]c(N)nc32)C[C@@H]1O]

or a pharmaceutically acceptable salt thereof.
25. A compound having the formula:

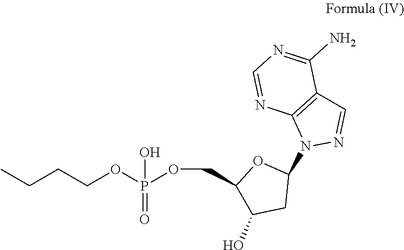 [SMILES: CCCCOP(=O)(O)OC[C@H]1O[C@@H](n2ncc3c(N)ncnc32)C[C@@H]1O]

or a pharmaceutically acceptable salt thereof
26. A compound having the formula:

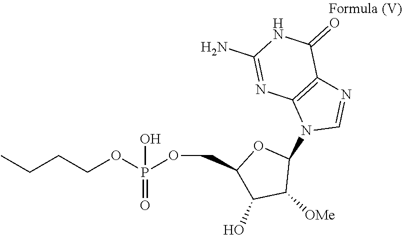 [SMILES: CCCCOP(=O)(O)OC[C@H]1O[C@@H](n2cnc3c(=O)[nH]c(N)nc32)[C@H](OC)[C@@H]1O]

or a pharmaceutically acceptable salt thereof.
27. A compound having the formula:

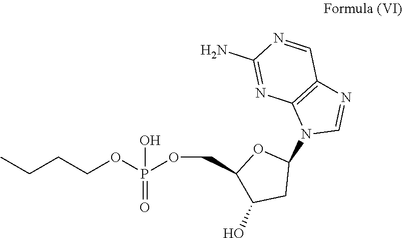 [SMILES: CCCCOP(=O)(O)OC[C@H]1O[C@@H](n2cnc3cnc(N)nc32)C[C@@H]1O]

or a pharmaceutically acceptable salt thereof.
28. A pharmaceutical composition comprising the compound according to any one of clauses 1 to 27, or a pharmaceutically acceptable salt thereof, and one or more pharmaceutically acceptable carriers, excipients, diluents, or a mixture of two or more thereof.
29. A method of treating an infection in a patient in need thereof, the method comprising administering an effective amount of a compound according to any one of clauses 1 to 27, or a pharmaceutically acceptable salt thereof, to the patient.
30. A method of treating an infection of at least one of a wound or an ulcer in a patient in need thereof, the method comprising administering an effective amount of a compound according to any one of clauses 1 to 27, or a pharmaceutically acceptable salt thereof, to the patient.
31. A method of treating an infection of a diabetic foot ulcer in a patient in need thereof, the method comprising administering an effective amount of a compound according to any one of clauses 1 to 27, or a pharmaceutically acceptable salt thereof, to the patient.
32. A method of treating a bladder and/or a urinary tract infection in a patient in need thereof, the method comprising administering an effective amount of a compound according to any one of clauses 1 to 27, or a pharmaceutically acceptable salt thereof, to the patient.
33. A method of treating a lung infection in a patient in need thereof, the method comprising administering an effective amount of a compound according to any one of clauses 1 to 27, or a pharmaceutically acceptable salt thereof, to the patient.
34. The method of clause 33, wherein the lung infection arises from a pulmonary condition.
35. The method of clause 34, wherein the pulmonary condition is selected from the group consisting of genetic conditions, acquired conditions, primary conditions, secondary conditions, asthma, chronic obstructive pulmonary disease, cystic fibrosis, bronchiolitis, pneumonia, bronchitis, emphysema, adult respiratory distress syndrome, allergies, lung cancer, small cell lung cancer, primary lung cancer, metastatic lung cancer, bronchiestasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, and conditions caused by any one or combination of the above.
36. The method of clause 34, wherein the pulmonary condition arises from a coronavirus.
37. The method of clause 36, wherein the coronavirus is selected from the group consisting of an alphacoronavirus, a betacoronavirus, a gammacoronavirus, a deltacoronavirus, and an omicroncoronavirus, or combinations thereof.
38. The method of clause 36, wherein the coronavirus is selected from the group consisting of porcine epidemic diarrhea virus (PEDv), scotophilus bat coronavirus 512, bat coronavirus CDPHE15, BtRF-Alpha-CoV HuB-2013, bat coronavirus HKU10, miniopterus bat coronavirus HKU8, miniopterus bat coronavirus 1, Nyctalus velutinus alphacoronavirus SC-2013, Pipistrellus kuhli coronavirus 3398, Myotis ricketti alphacoronavirus Sax-2011, HumCoV 229E, 229E-related bat coronavirus, camel alphacoronavirus, alpaca respiratory coronavirus, HumCoV NL63, NL63-related Bat-CoV BtKYNL63-9b, HKU2, SADSr-CoV, SADS-CoV, Lucheng Rn rat coronavirus2, FIPV, TGEV, PRCV, alphacoronavirus 1, mink coronavirus 1, FRCoV-NL-2010, Sorex araneus coronavirus T14, Suncus murinus coronavirus X74, HumCoV OC43, HumCoV OC43 isolate TNP F1778 2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYNO2, human SARS-CoV-2, cat-CoV, tiger-CoV, dog-CoV, mink-CoV, GD pangolin-CoV, bat Hp-betacoronavirus Zhejiang2013, peafowl IBV, avian coronavirus 9203, fowl IBV, avian coronavirus, duck-CoV, partridge IBV, goose coronavirus CB17, beluga whale coronavirus SW1, HKU22, HKU19, HKU20, HKU21, HKU11, HKU12, HKU16, HKU27, HKU28, HKU29, HKU18, HKU13, HKU30-UAE, HKU30-Poland, HKU17-USA, HKU17-China, AlCCoV, and HKU15, or combinations thereof.
39. The method of clause 36, wherein the coronavirus is a betacoronavirus selected from the group consisting of HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYNO2, human SARS-CoV-2, cat-CoV, tiger-CoV, dog-CoV, mink-CoV, GD pangolin-CoV, and bat Hp-betacoronavirus Zhejiang2013, or combinations thereof.
40. A method of treating cystic fibrosis in a patient in need thereof, the method comprising administering an effective amount of a compound according to any one of clauses 1 to 27, or a pharmaceutically acceptable salt thereof, to the patient.
41. A method of treating pneumonia in a patient in need thereof, the method comprising administering an effective amount of a compound according to any one of clauses 1 to 27, or a pharmaceutically acceptable salt thereof, to the patient.
42. The method of clause 41, wherein the pneumonia is ventilator acquired pneumonia.
43. A method of treating an infection in a burn wound in a patient in need thereof, the method comprising administering an effective amount of a compound according to any one of clauses 1 to 27, or a pharmaceutically acceptable salt thereof, to the patient.
44. A method of treating otitis externa in a patient in need thereof, the method comprising administering an effective amount of a compound according to any one of clauses 1 to 27, or a pharmaceutically acceptable salt thereof, to the patient.
45. A method of treating bacterial vaginosis in a patient in need thereof, the method comprising administering an effective amount of a compound according to any one of clauses 1 to 27, or a pharmaceutically acceptable salt thereof, to the patient.
46. A method of treating impetigo in a patient in need thereof, the method comprising administering an effective amount of a compound according to any one of clauses 1 to 27, or a pharmaceutically acceptable salt thereof, to the patient.
47. A method of treating oral mucositis in a patient in need thereof, the method comprising administering an effective amount of a compound according to any one of clauses 1 to 27, or a pharmaceutically acceptable salt thereof, to the patient.

### EXAMPLES

Examples related to the present disclosure are described below. In most cases, alternative techniques can be used. The examples are intended to be illustrative and are not limiting or restrictive of the scope of the invention as set forth in the claims.

### Example 1

### Synthesis of ((2R,3S,5R)-5-(6-amino-9H-purin-9-yl)-3-hydroxytetrahydrofuran-2-yl)methyl butyl hydrogen phosphate (LAI-017)

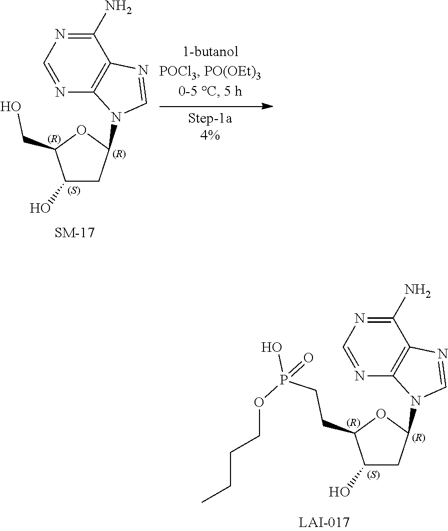

Phosphorous oxychloride (POCl₃) (3.72 ml, 39.80 mmole) was added to a stirred solution of (2R,3S,5R)-5-(6-amino-9H-purin-9-yl)-2-(hydroxymethyl)tetrahydrofuran-3-ol (SM-17) (10.00 g, 39.80 mmole) in triethyl phosphate (100 mL) at 0° C. under N₂ atmosphere with 4A molecular sieve (w/w) and stirred for 2 h. Butan-1-ol (5.46 mL, 59.70 mmole) was added and the reaction mixture was stirred at 0° C. for 5 h. After completion of reaction, which was monitored by LCMS, the reaction mixture was poured into diethyl ether (1000 mL) and 10% saturated sodium bicarbonate solution (60 mL). The aqueous layer was acidified by 1N HCl (pH~2.0) and lyophilized. After completion of lyophilization crude reaction mixture was subjected to reverse phase column chromatography (C18, SiO₂, 0-15% acetonitrile in water & 0.1% formic acid in water) and fractions containing product were lyophilized to afford pure LAI-017 (0.572 g, 4%) as a white solid.

¹H NMR 400 MHz, DMSO-d6: δ 8.31 (s, 1H), 8.14 (s, 1H), 1.35 (s, 1H), 6.38-6.35 (t, J=6.8 Hz, 1H), 5.48 (bs, 1H), 4.46-4.45 (m, 1H), 4.09-3.96 (m, 3H), 3.94-3.77 (m, 2H), 2.80-2.77 (m, 1H), 2.34-2.29 (m, 1H), 1.49-1.44 (m, 2H), 1.28-1.27 (m, 2H), 0.84-0.80 (m, 3H). ¹³C NMR 100 MHz, DMSO-d6: 13.42, 18.20, 31.78, 38.88, 65.50, 65.56, 65.87, 65.92, 70.60, 83.35, 85.30, 85.38, 119.03, 139.59, 148.98, 151.68, 155.37. ³¹P NMR 162 MHz, DMSO-d6: −1.260.

Elemental Analysis: N: 17.57%; C: 42.08%; H: 5.95%. LCMS (m/z): 388.23 [M-H]⁺.

### Example 2

### Synthesis of ((2R,3S,5R)-5-(2-amino-1,9-dihydro-6H-purin-6-one-9-yl)-3-hydroxytetrahydrofuran-2-yl)-methyl butyl hydrogen phosphate (LAI-R-4)

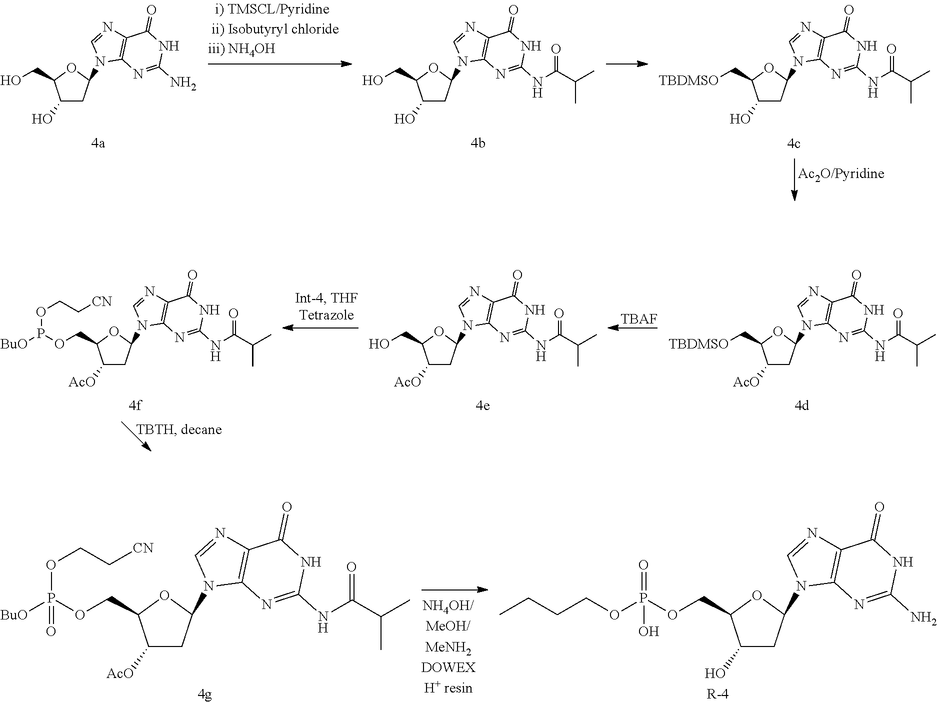

Step-1: Synthesis of compound 4b: Compound-4a (5 g, 0.0187 mol) was dried by co-evaporation of with dry pyridine (3×50 mL) in vacuum. The residue was suspended in dry pyridine (125 mL), and TMSCl (15.2 g, 0.1399 mol) was added drop wise at 0° C. under nitrogen atmosphere. The resulting reaction mixture was stirred at ambient temperature for 2 h, and then isobutyryl chloride (5.98 g, 0.056 mol) was added dropwise over 15 min at 0° C. The mixture was allowed to warm to room temperature and was stirred for 3 h. After completion of reaction, the reaction mass was quenched with water (250 mL) followed by aq NH₄OH (32.5 ml. The mixture was stirred for 15 min and washed with dichloromethane (100 mL). The aqueous layer was evaporated under reduced pressure at 45° C. to afford crude compound. The crude compound was co-evaporated with toluene 3 times and the crude compound was purified by column chromatography using 100-200 silica mesh, with the compound eluted using 10% MeOH in dichloromethane. The pure fractions were combined and dried over anhydrous Na₂SO₄, then filtered, and the solvent was evaporated under reduced pressure to afford compound 4b (4.3 g, 68.25%) as an off-white solid.

¹H NMR 400 MHz, DMSO-d6: δ 1.13 (d, J=6.94 Hz, 6H), 2.29 (dd, J=12.97, 6.03, 3.29 Hz, 1H), 2.54-2.62 (t, 1H), 2.76-2.83 (m, 1H), 3.47-3.63 (t, 2H), 3.82-3.89 (m, 1H), 4.39 (dt, J=5.57, 2.88 Hz, 1H), 6.22 (t, J=6.76 Hz, 1H), 8.24 (s, 1H), 11.71 (br s, 1H), 12.07 (br s, 1H). LCMS (m/z): 338.14 [M-H]⁺.

Step-2: Synthesis of compound 4c: To a solution of compound 4b (4.2 g, 0.0124 mol) in pyridine (42 mL) were added imidazole (2.12 g, 0.0311 mol) followed by TBDMSCl (2.25 g, 0.0149 mol) at room temperature dropwise at 0° C. The reaction mixture was stirred at room temperature for 3 h and the progress of the reaction was monitored by TLC. After completion of the reaction, pyridine was evaporated under reduced pressure at 45° C. The crude compound was co-evaporated with toluene 3 times and then dissolved in ethyl acetate (84 mL) and washed with water (42 mL) and brine (42 mL). The organic layer was dried over anhydrous Na₂SO₄, then filtered, and the solvent evaporated under reduced pressure to afford crude compound. The crude compound was purified by column chromatography using 100-200 silica mesh eluting with 5 MeOH in dichloromethane. The pure fractions were collected and evaporated under reduced pressure to afford compound 4c (4.5 g, 80.21%) as a light brown solid.

¹H NMR 400 MHz, DMSO-d6: δ 0.03 (s, J=2.56 Hz, 6H), 0.84 (s, 9H), 1.12 (d, J=6.94 Hz, 6H), 1.88-1.95 (t, 1H), 2.54 (br s, 1H), 2.70-2.89 (m, 1H), 3.77 (dd, J=4.20, 2.38 Hz, 2H), 4.08 (br d, J=1.46 Hz, 1H), 5.30 (br d, J=5.85 Hz, 1H), 6.21 (dd, J=8.59, 5.66 Hz, 1H), 8.17 (s, 1H), 11.67 (br s, 1H), 12.07 (br s, 1H). LCMS (m/z): 452.23 [M-H]⁺.

Step-3: Synthesis of compound 4d: To a solution of compound 4c (4.5 g, 0.0099 mol) and pyridine (27 mL) and dichloromethane (13.5 mL) at room temperature, acetic anhydride (2.9 g, 0.0284 mol) was added drop-wise at 0° C. The reaction mixture was stirred at room temperature for 6 h and the progress of the reaction was monitored by TLC. After completion of the reaction, the reaction mass was diluted with ethyl acetate (180 mL) and washed with saturated NaHCO₃ (3×45 mL) solution and brine (45 mL). The organic layer was dried over anhydrous Na₂SO₄, then filtered, and the solvent was evaporated under reduced pressure to afford crude compound. The crude compound was purified by column chromatography using silica 100-200 mesh, with the compound eluted using 3-5% methanol in dichloromethane. The pure fractions were collected and evaporated under reduced pressure to afford compound 4d (4 g, 81.63%) as a light brown solid.

¹H NMR 400 MHz, DMSO-d6: δ 0.03 (s, J=2.56 Hz, 6H) 0.84 (s, 9H), 1.12 (d, J=6.94 Hz, 6H), 1.88-1.95 (t, 1H), 2.08 (s, 3H), 2.54 (br s, 1H), 2.70-2.89 (m, 1H), 3.77 (dd, J=4.20, 2.38 Hz, 2H), 4.08 (br d, J=1.46 Hz, 1H), 5.30 (br d, J=5.85 Hz, 1H), 6.21 (dd, J=8.59, 5.66 Hz, 1H), 8.17 (s, 1H), 11.67 (br s, 1H), 12.07 (br s, 1H). LCMS (m/z): 494.24 [M-H]⁺.

Step-4: Synthesis of compound 4e: To a solution of compound 4d (4 g, 0.00811 mol) in THF (40 mL), tetrabutyl ammonium fluoride (40.56 ml, 1M) was added dropwise at 0° C. The reaction mixture was stirred at room temperature for 12 h and the progress of the reaction was monitored by TLC. After completion of the reaction, the solvent was evaporated under reduced pressure at 45° C. to afford crude compound. The crude compound was dissolved in ethyl acetate (80 mL) and the organic layer was washed with brine solution (40 mL) and water (40 mL) and then dried over anhydrous Na₂SO₄ and filtered. The solvent was evaporated under reduced pressure to afford crude compound. The crude compound was purified by column chromatography using silica 100-200 mesh, with the compound eluted using 3-5% methanol in dichloromethane. The pure fractions were collected and evaporated under reduced pressure to afford compound 4e (2 g, 65.14%) as an off-light brown solid.

¹H NMR 400 MHz, DMSO-d6: δ 1.12 (d, J=6.97 Hz, 6H), 1.95-2.00 (m, 1H), 2.08 (s, 3H), 2.44 (br d, J=4.40 Hz, 1H), 2.70-2.88 (m, 1H), 3.59 (t, J=4.95 Hz, 2H), 4.01-4.07 (m, 1H), 5.13 (t, J=5.50 Hz, 1H), 5.31 (br d, J=5.50 Hz, 1H), 6.21 (dd, J=8.99, 5.69 Hz, 1H), 8.28 (s, 1H), 11.70 (s, 1H), 12.09 (s, 1H). LCMS (m/z): 280.15 [M-H]⁺.

Steps-5 and 6: Synthesis of compound 4g: To a solution of compound 4e (1.5 g, 0.00395 mol) in dichloromethane (15 mL), was added DIPEA (2.55 g, 0.0197 mol), followed by tetrazole (0.694 g, 0.00989) at 0° C. To the above resulting reaction mixture, Int-4 (2.71 g, 0.00989 mol) in dichloromethane (15 mL) was added dropwise at 0° C. and the resulting reaction mixture was stirred for 16 h at room temperature. After consumption of the starting material, the reaction mass was evaporated under reduced pressure. The crude product was dissolved in THF (15 mL), and tert-butyl hydroperoxide in decane (2.37 mL, 5M) was added drop wise at 0° C. and stirred for 3 h at room temperature. The progress of the reaction was monitored by TLC, and after completion of the reaction, the reaction mixture was quenched with ice water and diluted with ethyl acetate (50 mL). The organic layer was separated and washed with saturated brine solution and water, dried over anhydrous Na₂SO₄ and then filtered. The solvent was evaporated under reduced pressure to afford crude compound. The crude compound was purified by column chromatography using silica 100-200 mesh, with the compound eluted using 1% MeOH in ethyl acetate. The pure fractions were collected and evaporated under reduced pressure to afford compound 4g (1.5 g, 66.96%) as wine-red colored liquid.

¹H NMR 400 MHz, DMSO-d6: δ 0.79-0.93 (t, 3H) 1.08-1.38 (m, 10H), 1.46-1.62 (m, 2H), 1.99 (s, 3H), 2.76 (br dd, J=12.84, 5.87 Hz, 1H), 2.89 (br d, J=5.50 Hz, 2H), 3.92-4.31 (m, 7H) 5.35 (br s, 1H), 6.24 (br d, J=6.60 Hz, 1H), 8.23 (br s, 1H), 11.64 (br s, 1H), 12.09 (br s, 1H). LCMS (m/z): 569.20 [M-H]⁺.

Step-7: Synthesis of compound LAI-R-4: To a solution of compound 4g (1.5 g, 0.0026 mol) in MeOH (15 mL) was added aq. ammonia (10.23 g, 0.29 mol) and MeNH₂ (165 mL, 2M) at 0° C. The resulting reaction mixture was stirred for 16 h at room temperature and the progress of the reaction was monitored by TLC. After completion of the reaction, the solvent was evaporated under reduced pressure. The aqueous layer was washed with dichloromethane (3×10 mL) and then passed through DOWEX-H⁺ resin. The eluate was evaporated under reduced pressure to afford ((2R,3S,5R)-5-(2-amino-1,9-dihydro-6H-purin-6-one-9-yl)-3-hydroxytetrahydrofuran-2-yl)-methyl butyl hydrogen phosphate (LAI-R-4) (0.07 g, 6.6%).

¹H NMR 400 MHz, DMSO-d6: δ 0.85 (t, J=7.34 Hz, 3H), 1.28 (dd, J=14.98, 7.23 Hz, 2H), 1.37-1.50 (m, 2H), 2.12-2.24 (m, 1H), 2.56-2.70 (m, 1H), 3.59 (q, J=6.36 Hz, 2H), 3.67-3.76 (m, 1H), 3.81-3.95 (m, 2H), 4.50 (bs, 1H), 5.64 (bs, 1H), 6.10 (br t, J=6.60 Hz, 1H), 6.58 (bs, 1H), 7.89 (s, 1H), 10.48 (s, 1H). LCMS (m/z): 404.13 [M-H]⁺.

### Example 3

### Synthesis of ((2R, 3S, 5R)-5-(4-amino-1H-pyrazolo [3,4-d]pyrimidine-1-yl)-3-hydroxytetra hydrofuran-2-yl)methyl butyl hydrogen phosphate (LAI-018)

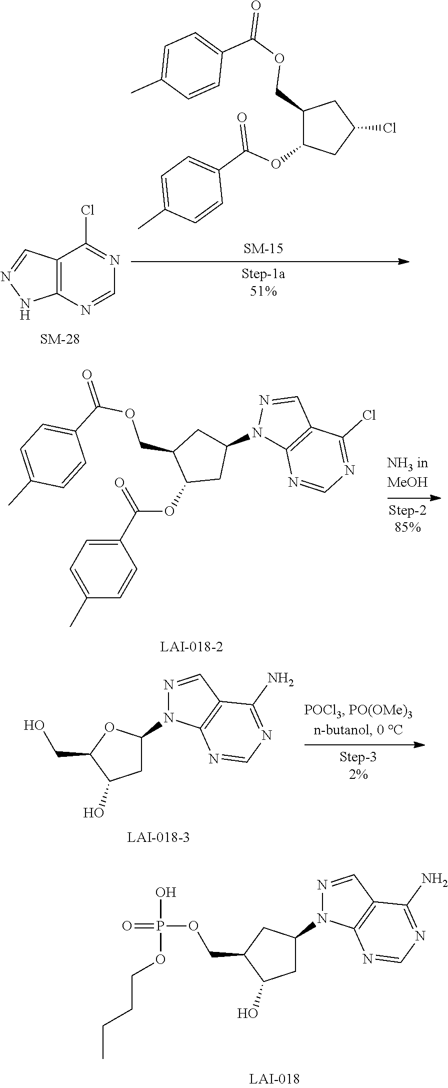

Step-1: Synthesis of (2R,3S,5R)-5-(4-chloro-1H-pyrazolo[3,4-d]pyrimidin-1-yl)-2-(((4-methylbenzoyl)oxy)methyl)tetrahydrofuran-3-yl 4-methyl benzoate: To a stirred solution of 4-chloro-1H-pyrazolo[3,4-d]pyrimidine (10 g, 64.68 mmole) in acetonitrile (150 mL), NaH (3.10 g, 77.61 mmole) was added portion wise at 0° C. for 15 min. 2-Deoxy-a-D-erythro-pentofuranosyl-3,5-bis(4-methylbenzoate) chloride (20.12 g, 51.54 mmole) was added and the reaction mixture was stirred at room temperature for 16 h under nitrogen. After completion of the reaction, which was monitored by LCMS, the reaction mixture was quenched by ice-water (100 mL) and extracted with dichloromethane. The organic layer was dried over sodium sulphate, filtered, and evaporated to obtained crude product which was purified by column chromatography (60-120, Silica). The product was eluted at 30-40% ethyl acetate in petroleum ether and the fractions were concentrated to afford (2R,3S,5R)-5-(4-chloro-1H-pyrazolo[3,4-d]pyrimidin-1-yl)-2-(((4-methylbenzoyl)oxy)methyl)tetrahydrofuran-3-yl-4-methyl benzoate (LAI-018-2) (17 g, 51% yield).

Step-2: Synthesis of (2R, 4S, 5R)-5-(4-amino-1H-pyrazolo [3,4-d]pyrimidine-1-yl)-2-(hydroxyl methyl) tetrahydrofuran-3-ol: LAI-018-2 (5 g, 98.81 mmole) was dissolved in NH₃ in methanol (50 mL) and the reaction mixture was stirred at 60° C. for 16 h. After completion of the reaction, which was monitored by LCMS, the reaction mixture was concentrated and recrystallized by 10% methanol in dichloromethane (20 mL) to afford (2R, 4S, 5R)-5-(4-amino-1H-pyrazolo [3,4-d]pyrimidine-1-yl)-2-(hydroxymethyl) tetrahydrofuran-3-ol (LAI-018-3) (2.1 g, 85% yield).

Step: 3 Synthesis of ((2R, 3S, 5R)-5-(4-amino-1H-pyrazolo [3,4-d]pyrimidine-1-yl)-3-hydroxytetra hydrofuran-2-yl)methyl butyl hydrogen phosphate: Phosphorous oxychloride (POCl₃) (3.55 ml, 37.99 mmole) was added to a stirred solution of LAI-018-3 (5.00 g, 19.91 mmole) in trimethyl phosphate (50 mL) at 0° C. under N₂ atmosphere with 4A molecular sieve (w/w) and stirred at 0° C. for 2 h. Butan-1-ol (5.466 mL, 59.73 mmole) was added and the reaction mixture was stirred at 0° C. for 5 h. After completion of the reaction, which was monitored by LCMS, the reaction mixture was poured into diethyl ether (1000 mL) and 10% saturated sodium bicarbonate solution (60 mL). The aqueous layer was acidified by 1N HCl (30 mL) at pH~2.0 and lyophilized. After completion of lyophilization, the crude reaction mixture was subjected to reverse phase column chromatography (C18, Silica) where the product was eluted at 0-15% acetonitrile in water (using 0.1% formic acid in water) and the fractions were lyophilized to afford pure LAI-018 (210 mg, 2% yield) as a white solid.

¹H NMR 400 MHz, DMSO-d6: δ 9.22 (d, J=12.9 Hz, 1H), 8.39 (d, J=5.4 Hz, 1H), 6.82 (d, J=5.5 Hz, 1H), 6.13 (m, J=5.6 Hz, 1H), 5.40 (d, J=15.7 Hz, 1H), 5.27 (d, J=10.5 Hz, 1H), 4.91 (d, J=5.2 Hz, 1H), 3.88 (m, J=8.5 Hz, 1H), 1.94 (s, 1H), 1.72 (t, J=5.4 Hz, 1H), 1.39 (d, J=5.9 Hz, 1H), 1.24 (s, 1H), 0.94 (s, 1H). ¹³C NMR 100 MHz, DMSO-d6: 13.44, 18.20, 31.76, 31.83, 37.78, 65.34, 65.40, 66.25, 66.31, 70.85, 83.61, 84.95, 85.03, 100.36, 133.42, 153.63, 155.61, 157.63. ³¹P NMR 162 MHz, DMSO-d6: −1.617. Elemental Analysis: N: 16.70%; C: 40.40%; H: 5.86%. LCMS (m/z): 388.26 [M-H]⁺.

### Example 4

### Synthesis of ((2R,3R,4R,5R)-5-(2-amino-1,9-dihydro-6H-purin-6-one-9-yl)-4-methoxy-3-hydroxytetrahydrofuran-2-yl)-methyl butyl hydrogen phosphate (LAI-R-8)

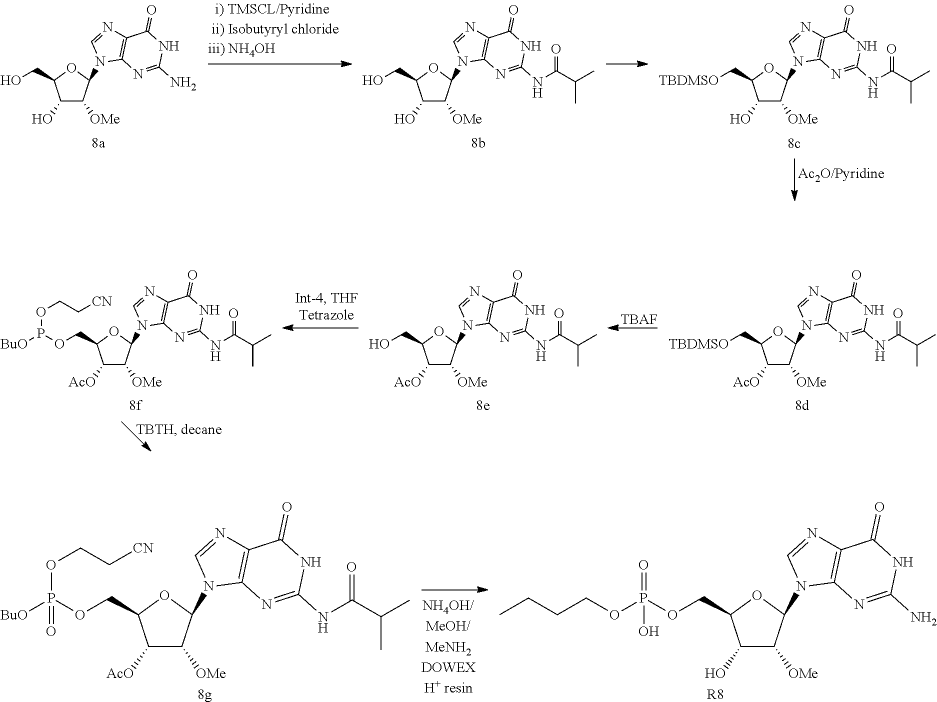

Step-1: Synthesis of compound 8b: Compound-8a (5 g, 0.0168 mol) was dried by co-evaporation with dry pyridine (3×50 mL) in vacuum. The residue was suspended in dry pyridine (125 mL) under nitrogen atmosphere, and TMSCl (13.7 g, 0.126 mol) was added drop wise at 0° C. The solution was stirred at ambient temperature for 2 hours, then cooled to 0° C. and isobutyryl chloride (5.38 g, 0.050 mol) was added dropwise over 15 min at 0° C. The reaction mixture was allowed to warm to room temperature, stirred for 3 hours, and then cooled to 0° C. before quenching by the addition of water (15 mL). After stirring for 5 min at 0° C. and then 5 min at room temperature, aq. NH₄OH (32.5 mL) was added. After stirring for an additional 15 min at room temperature, the mixture was diluted with water (250 mL) and washed with dichloromethane (100 mL). The aqueous layer was evaporated under reduced pressure at 45° C. to afford crude compound. The crude compound was co-evaporated with toluene 3 times and then purified by column chromatography by using 100-200 silica mesh, with the compound eluted using 10% MeOH in dichloromethane. The pure fractions were collected and evaporated under reduced pressure to afford compound 8b (4.3 g, 69.69%) as an off-white solid.

¹H NMR 400 MHz, DMSO-d6: δ 1.13 (d, J=6.94 Hz, 6H) 2.54-2.62 (t, 1H) 3.22-3.34 (s, 3H) 3.47-3.63 (t, 2H) 3.82-3.89 (m, 2H) 4.39 (dt, J=5.57, 2.88 Hz, 1H) 6.22 (t, J=6.76 Hz, 1H) 8.24 (s, 1H) 11.71 (bs, 1H) 12.07 (bs, 1H). LCMS (m/z): 368.15 [M-H]⁺.

Step-2: Synthesis of compound 8d: To a stirred solution of compound 8b (4.0 g, 0.0109 mol) in pyridine (40 mL), imidazole (0.89 g, 0.013 mol) and then TBDMSCl (1.97 g, 0.013 mol) were added dropwise at 0° C. The reaction mixture was stirred at room temperature for 3 h and the progress of the reaction was monitored by TLC. After completion of the reaction, dichloromethane (16 mL) was added at room temperature, followed by acetic anhydride (3.169 g, 0.031 mol), which was added drop wise at 0° C. The reaction mixture was stirred at room temperature for 6 h and the progress of the reaction was monitored by TLC. After completion of the reaction, the reaction mass was diluted with ethyl acetate (160 mL) and washed with saturated NaHCO₃ (3×40 mL) and brine (40 mL). The organic layer was dried over anhydrous Na₂SO₄, then filtered, and the solvent was evaporated under reduced pressure to afford crude compound. The crude compound was purified by column chromatography using silica 100-200 mesh, with the compound eluted using 3-5% methanol in dichloromethane. The pure fractions were collected and evaporated under reduced pressure to afford compound 8d (2.5 g, 43.93%) as a light brown solid.

¹H NMR 400 MHz, DMSO-d6: δ 0.08 (d, J=6.85 Hz, 6H), 0.89 (s, 9H), 1.13 (d, J=6.85 Hz, 6H), 2.11-2.16 (m, 3H), 2.77 (dt, J=13.69, 6.85 Hz, 1H), 3.81-4.35 (m, 5H), 4.66 (dd, J=7.34, 5.38 Hz, 2H), 5.41 (dd, J=5.38, 2.45 Hz, 1H), 5.87-5.91 (m, 1H), 8.29 (s, 1H) 11.63 (br s, 1H), 12.10 (br s, 1H). LCMS (m/z): 524.25 [M-H]⁺.

Step-3: Synthesis of compound 8e: To a stirred solution of compound 8d (2.5 g, 0.00478 mol) in THF (25 mL), was added TBAF (23.9 ml, 1M) dropwise at 0° C. The reaction mixture was stirred at room temperature for 12 h and the progress of the reaction was monitored by TLC. After completion of the reaction, the solvent was evaporated under reduced pressure at 45° C. to afford crude compound. The crude compound was dissolved in ethyl acetate (50 mL) and washed with brine (25 mL) and water (25 mL). The organic layer was dried over anhydrous Na₂SO₄, then filtered, and the solvent was evaporated under reduced pressure to afford crude compound. The crude compound was purified by column chromatography using silica 100-200 mesh, with the compound eluted using 3-5% methanol in dichloromethane. The pure fractions were collected and evaporated under reduced pressure to afford compound 8e (1.7 g, 87.17%) as an off-white solid.

¹H NMR 400 MHz, DMSO-d6: δ 1.13 (d, J=6.85 Hz, 6H), 2.12 (s, 3H), 2.77 (dt, J=13.57, 6.66 Hz, 1H), 3.25 (s, 3H) 3.63 (t, J=4.65 Hz, 2H), 4.13 (d, J=1.47 Hz, 1H), 4.57 (dd, J=7.58, 5.14 Hz, 1H), 5.30 (t, J=5.38 Hz, 1H), 5.41-5.46 (m, 1H), 5.87 (d, J=7.34 Hz, 1H), 8.33 (s, 1H), 11.66 (s, 1H), 12.10 (s, 1H). LCMS (m/z): 410.16 [M-H]⁺.

Steps-5 and 6: Synthesis of compound 8g: To a stirred solution of compound 8e (1.7 g, 0.0041 mol) in dichloromethane (17 mL), DIPEA (2.686 g, 0.02078 mol) and then tetrazole (0.578 g, 0.0082) were added at 0° C. Int-4 (2.84 g, 0.01036 mol) in dichloromethane (17 mL) was added drop wise at 0° C. and the resulting reaction mixture was stirred for 16 h at room temperature. After consumption of the starting material, the reaction mass evaporated under reduced pressure. The crude product was dissolved in THF (17 mL), and tert-butyl hydroperoxide in decane (2.5 mL, 5M) was added dropwise at 0° C. and stirred for 3 h at room temperature. The progress of the reaction was monitored by TLC, and after completion of the reaction, was quenched with ice water and diluted with ethyl acetate (50 mL). The organic layer was separated and washed with saturated brine (20 mL) and water (20 mL), dried over anhydrous Na₂SO₄, then filtered, and the solvent was evaporated under reduced pressure to afford crude compound. The crude compound was purified by column chromatography using silica 100-200 mesh, with the compound eluted using 1% MeOH in ethyl acetate. The pure fractions were collected and evaporated under reduced pressure to afford compound 8g (1.8 g, 79.64%) as wine-red colored liquid.

¹H NMR 400 MHz, DMSO-d6: δ 0.75-0.96 (m, 3H), 1.05-1.18 (m, 6H), 1.20-1.39 (m, 4H), 2.09-2.19 (m, 3H), 2.81-2.95 (m, 2H), 3.13-3.21 (m, 3H), 3.92-4.06 (m, 2H), 4.06-4.19 (m, 2H) 4.24-4.38 (m, 3H), 4.62 (br dd, J=12.23, 6.36 Hz, 1H), 5.46 (br dd, J=5.14, 2.69 Hz, 1H), 5.92 (br d, J=7.34 Hz, 1H), 8.26 (d, J=3.91 Hz, 1H), 11.59 (s, 1H), 12.05-12.17 (m, 1H). LCMS (m/z): 599.22 [M-H]⁺.

Step 7: Synthesis of LAI-R-8: To a stirred solution of compound 8g (0.9 g, 0.00165 mol) in MeOH (9 ml), aq. NH₄OH (7.22 g, 0.206 mol) followed by MeNH₂ (103.2 mL, 2M) were added at 0° C. The resulting reaction mixture was stirred for 16 h at room temperature and the progress of the reaction was monitored by TLC. After completion of the reaction, the solvent was evaporated under reduced pressure. The crude was diluted with water (10 mL) and washed with dichloromethane (3×10 mL). The aqueous layer was passed through DOWEX-H⁺ resin and the eluate was evaporated under reduced pressure to afford ((2R,3R,4R,5R)-5-(2-amino-1,9-dihydro-6H-purin-6-one-9-yl)-4-methoxy-3-hydroxytetrahydrofuran-2-yl)-methyl butyl hydrogen phosphate (LAI-R-8) (233 mg, 35.84%).

¹H NMR 400 MHz, DMSO-d6: δ 0.81-0.92 (t, 3H), 1.26-1.36 (m, 2H), 1.49-1.58 (m, 2H), 3.34 (s, 3H), 3.82-3.90 (m, 2H), 3.99-4.13 (m, 3H), 4.19 (t, J=5.38 Hz, 1H), 4.28-4.32 (m, 1H), 5.84 (d, J=5.87 Hz, 1H), 6.56 (bs, 2H), 8.02 (s, 1H), 10.75 (bs, 1H). LCMS (m/z): 434.14 [M-H]⁺.

### Example 5

### Synthesis of ((2R,3S,5R)-5-(2-amino-9H-purin-9-yl)-3-hydroxytetrahydrofuran-2-yl)-methyl butyl hydrogen phosphate (LAI-020)

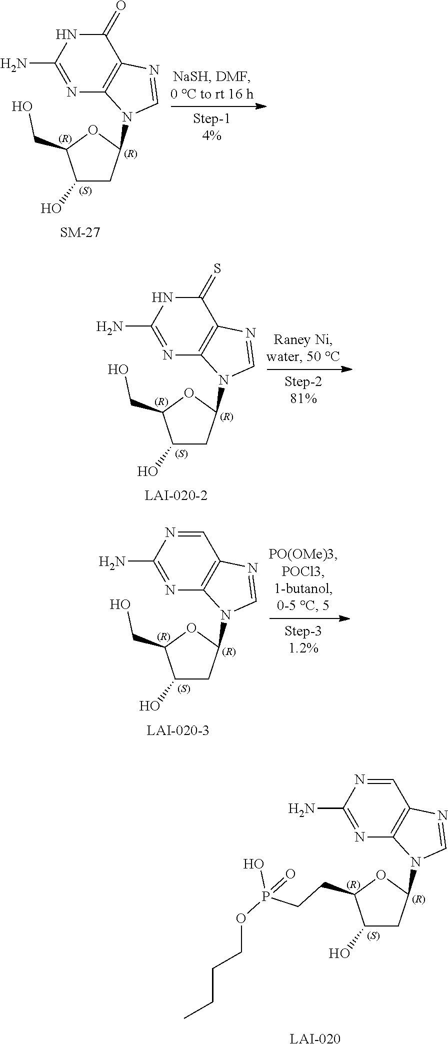

Step-1: Synthesis of 2-amino-9-((2R,4S,5R)-4-hydroxy-5 (hydroxymethyl)tetrahydrofuran-2-yl)-1,9-dihydro-6H-purine-6-thione: 2-amino-9-((2R,4S,5R)-4-hydroxy-5-(hydroxymethyl)tetrahydrofuran-2-yl)-1,9-dihydro-6H-purin-6-one (SM-27) (20 g, 74.2 mmol) was dried three times by evaporation of pyridine and the dried compound was suspended in 1.5 L of pyridine and cooled at 0° C. under a nitrogen atmosphere. Trifluoracetic acid (82.6 ml) was added dropwise and the reaction mixture was stirred for 40 min. A suspension of NaSH (124.9 g, 2.22 mmol) in DMF (2.26 L) was added to the mixture dropwise and the reaction was monitored by LCMS. After completion of the reaction, the reaction was quenched with 0.16 M 2000 ml ammonium bicarbonate solution, evaporated to dryness, washed with methanol, and filtered. The filtrate was concentrated to obtain the crude compound, which was purified by column chromatography and further triturated with ethyl acetate to obtain 2-amino-9-((2R,4S,5R)-4-hydroxy-5-(hydroxymethyl)tetrahydrofuran-2-yl)-1,9-dihydro-6H-purine-6-thione (LAI-020-2) as a brownish white solid (Yield: 9 g, 42%).

Step-2: (2R,3S,5R)-5-(2-amino-9H-purin-9-yl)-2-(hydroxymethyl)tetrahydrofuran-3-ol: To a stirred solution of LAI-020-2 (9 g, 31.8 mmol) in water (90 mL), Raney Ni (60 mL in water) was added, and the reaction mixture was stirred at 50° C. under N₂ atmosphere for 16 h. The reaction was monitored by TLC and LCMS. After completion of the reaction, the reaction mixture was filtered, and the filtrate was concentrated to obtain the crude compound, which was lyophilised to obtain the desired compound (LAI-020-3) (6.5 g) as an off-white solid.

Step-3: Synthesis of ((2R,3S,5R)-5-(2-amino-9H-purin-9-yl)-3-hydroxytetra-hydrofuran-2-yl)methyl butyl hydrogen phosphate: Phosphorous oxychloride (POCl₃) (2.28 ml, 24.4 mmole) was added to a stirred solution of LAI-020-3 (6.15 g, 24.49 mmol) in trimethyl phosphate (62 mL) at 0° C. under N₂ atmosphere with 4A molecular sieve (w/w), and the reaction mixture was stirred at 0° C. for 2 h. 1-Butanol (6.7 mL, 73.47 mmole) was added and the reaction mixture was stirred at 0° C. for 5 h. After completion of the reaction, which was monitored by LCMS, the reaction mixture was poured into diethyl ether (1000 mL) and 10% saturated sodium bicarbonate solution (60 mL). The aqueous layer was acidified by 1N HCl (30 mL) at pH~2.0 and lyophilized. After completion of lyophilization, the crude reaction mixture was subjected to reverse phase column chromatography (C₁₈, Silica) where the product was eluted at 0-11% acetonitrile in 0.1% formic acid in water. For further purification the crude compound was purified by preparative HPLC to afford LAI-020 (101 mg, 1.2%) as an off-white solid.

Preparative HPLC purification method: Column/dimensions: synergy polar C18 (19*250, 5 um); Mobile phase A:0.1% formic acid in water; Mobile phase B: acetonitrile; Gradient (Time/% B): 0/2, 2/2, 15/25, 15.1/98, 17/98, 17.1/2, 20/2; Flow rate: 18 ml/min.; Solubility: THF. 1H NMR 400 MHz, DMSO-d6: δ 8.60 (s, 1H), 8.23 (s, 1H), 6.61 (s, 2H), 6.31-6.27 (t, J=6.8 Hz, 1H), 5.45 (s, 1H), 4.42 (s, 1H), 4.12-4.09 (t, J=6.6 Hz, 1H), 3.99-3.95 (m, 2H), 3.84-3.79 (m, 2H), 2.70-2.67 (m, 1H), 2.32-2.31 (m, 1H), 1.55-1.45 (m, 2H), 1.33-1.21 (m, 2H), 0.90-0.88 (t, J=4.00 Hz, 3H). ¹³C NMR 100 MHz, DMSO-d6: 13.42, 18.18, 31.74, 31.81, 38.61, 65.68, 65.73, 65.99, 66.04, 70.56, 82.67, 85.17, 85.24, 126.99, 141.42, 148.01, 153.13, 159.65. ³¹P NMR 162 MHz, DMSO-d6: −1.222. Elemental Analysis: N: 15.86%; C: 40.41%; H: 5.78%. LCMS (m/z): 388.23 [M-H]⁺.

### Example 6

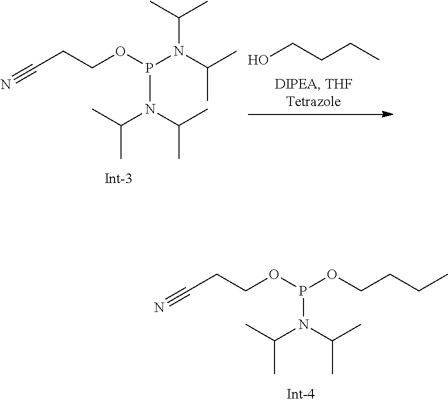

To a solution of Int-3 (100 g, 0.3222 mol) in THF (500 mL), DIPEA (138 mL, 0.8305 mol), tetrazole (24 g, 0.3222 mol), and then n-butanol (27.65 mL, 0.2990 mol) in THF (100 mL) were added dropwise at 0° C. over a period of 1 h. The reaction mixture was stirred at room temperature for 24 h and the progress of the reaction was monitored by TLC. After completion of the reaction, the solid was filtered off and the filtrate was evaporated under reduced pressure at 40° C. to afford crude compound. The crude compound was dissolved in ethyl acetate (250 mL) and washed with water (50 mL) and brine (50 mL) and then dried over anhydrous Na₂SO₄ and filtered. The solvent was evaporated under reduced pressure to afford crude compound. The crude compound was purified by column chromatography using basic alumina (Al₂O₃), with the compound eluted using 0-2% ethyl acetate in petroleum ether. All pure fractions were collected and concentrated to afford Int-4 (70 g, 76.92%) as a pale-yellow liquid.

¹H NMR 400 MHz, Chloroform-d: 6 0.85-1.00 (m, 3H), 1.11-1.24 (m, 12H), 1.33-1.50 (m, 2H), 1.52-1.76 (m, 2H), 2.59-2.72 (m, 2H), 3.52-3.72 (m, 3H), 3.74-3.92 (m, 3H). LCMS (m/z): 285.18 [M-H]⁺.

### Example 7

### Minimum Inhibitory Concentration

MIC testing is conducted using the broth (Mueller Hinton Broth) microdilution procedures relevant to each organism group as established by the Clinical and Laboratory Standards Institute (CLSI; 3-7). MIC plates are prepared in accordance with CLSI (3-7). To prepare the test plates, automated liquid handlers are used to conduct serial dilutions and liquid transfers. Automated liquid handlers utilized in this study include the Multidrop 384 (Labsystems, Helsinki, Finland) and the Biomek 2000 (Beckman Coulter, Fullerton CA). The solution is diluted to desired pH using 1 M Citric Acid or 1 M NaOH buffer. A standardized inoculum of each test organism is prepared per CLSI methods (3, 5-7) to equal a 0.5 McFarland standard in the appropriate media followed by an additional 1:20 dilution (1:10 for anaerobes).

The plates are then inoculated with 10 μL of the diluted 0.5 McFarland suspension using the Biomek 2000 from low to high drug concentration, resulting in a final concentration of approximately 5×10⁵ CFU/mL per well. An un-inoculated plate is incubated for the purpose of assessing solubility of the drug in the test media. The plates are stacked 3 to 4 high, covered with a sterile lid on the top plate, and incubated for 18 to 24 hours according to CLSI. The MIC is recorded as the lowest concentration of respective compound that inhibited visible growth of the organism. MIC data collected at pH 3 are summarized in TABLE 2. MIC data collected at pH 4-5 are summarized in TABLE 3.

TABLE 2

| | Organism | | | |
|---|---|---|---|---|
| | Staphylococcus | Escherichia | Pseudomonas | Candida |
| | aureus | coli | aeruginosa | albicans |
| | (43330) | (25922) | (27853) | (22972) |
| Drug | MIC (mg/mL) | | | |
| Nu-3 | 25 | 25 | 100 | 25 |
| Nu-8 | 100 | 100 | 100 | 100 |
| Nu-10 | 5 | 1 | 5 | 5 |
| LAI-017 | 25 | 25 | 5 | 5 |
| LAI-018 | 25 | 25 | 25 | 5 |
| LAI-020 | 25 | 25 | 5 | 25 |

TABLE 3

| | MIC (mg/mL) | | | |
|---|---|---|---|---|
| Organism | Nu-3 | Nu-8 | R-4 | R-8 |
| Escherichia coli (0102) | 200 | 50 | 50 | 100 |
| Escherichia coli (6260) | 100 | 50 | 100 | 100 |
| Escherichia coli (6098) | 100 | 50 | TBD | 100 |
| Escherichia coli (6258) | 100 | 50 | TBD | 100 |
| Escherichia coli (6259) | 200 | 25 | TBD | 100 |
| Pseudomonas aeruginosa (0103) | 50 | 25 | 25 | 100 |
| Pseudomonas aeruginosa (6160) | 50 | 25 | 50 | 100 |
| Pseudomonas aeruginosa (6234) | 100 | 12.5 | 25 | 50 |
| Pseudomonas aeruginosa (6326) | 100 | 25 | TBD | 100 |
| Pseudomonas aeruginosa (6328) | 200 | 25 | TBD | 100 |

### Example 8

### Treatment of Human Patient Having an Infection

A human patient is identified as having an infection. A pharmaceutical composition containing an effective amount of any compound described herein is administered in a manner consistent with the particular infection diagnosed. The patient is monitored until symptoms are alleviated or ameliorated, and the pharmaceutical composition may be administered one or more additional times if it is determined that such administration is necessary or helpful for treatment.

### Example 9

### Treatment of Human Patient Having an Infection of a Wound or an Ulcer

A human patient is identified as having an infection of of a wound or an ulcer. A pharmaceutical composition containing an effective amount of any compound described herein is administered to the patient, such as at the location of the wound or ulcer. The patient is monitored until symptoms are alleviated or ameliorated, and the pharmaceutical composition may be administered one or more additional times if it is determined that such administration is necessary or helpful for treatment.

### Example 10

### Treatment of Human Patient Having an Infection of a Lower Extremity

A human patient is identified as having an infection of a lower extremity. A pharmaceutical composition containing an effective amount of any compound described herein is administered to the patient, such as at the location of the infection of the lower extremity. The patient is monitored until symptoms are alleviated or ameliorated, and the pharmaceutical composition may be administered one or more additional times if it is determined that such administration is necessary or helpful for treatment.

### Example 11

### Treatment of Human Patient Having an Infection of a Diabetic Foot Ulcer

A human patient is identified as having a diabetic foot ulcer. A pharmaceutical composition containing an effective amount of any compound described herein is administered to the patient, such as at the location of the diabetic foot ulcer. The patient is monitored until symptoms are alleviated or ameliorated, and the pharmaceutical composition may be administered one or more additional times if it is determined that such administration is necessary or helpful for treatment.

### Example 12

### Treatment of Human Patient Having Bladder and/or a Urinary Tract Infection

A human patient is identified as having a bladder and/or a urinary tract infection. A pharmaceutical composition containing an effective amount of any compound described herein is administered to the patient, such as via a catheter. The patient is monitored until symptoms are alleviated or ameliorated, and the pharmaceutical composition may be administered one or more additional times if it is determined that such administration is necessary or helpful for treatment.

### Example 13

### Treatment of Human Patient Having a Lung Infection

A human patient is identified as having a lung infection. A pharmaceutical composition containing an effective amount of any compound described herein is administered to the patient, such as via a nebulizer. The patient is monitored until symptoms are alleviated or ameliorated, and the pharmaceutical composition may be administered one or more additional times if it is determined that such administration is necessary or helpful for treatment.

### Example 14

### Treatment of Human Patient Having a Lung Infection Arising from a Pulmonary Condition

A human patient is identified as having a lung infection arising from a pulmonary condition, such as genetic conditions, acquired conditions, primary conditions, secondary conditions, asthma, chronic obstructive pulmonary disease, cystic fibrosis, bronchiolitis, pneumonia, bronchitis, emphysema, adult respiratory distress syndrome, allergies, lung cancer, small cell lung cancer, primary lung cancer, metastatic lung cancer, bronchiestasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, or conditions caused by any one or combination of the above. A pharmaceutical composition containing an effective amount of any compound described herein is administered to the patient, such as via a nebulizer. The patient is monitored until symptoms are alleviated or ameliorated, and the pharmaceutical composition may be administered one or more additional times if it is determined that such administration is necessary or helpful for treatment.

### Example 15

### Treatment of Patient Having a Pulmonary Condition Arising from a Coronavirus

A patient is identified as having a pulmonary condition arising from a Coronavirus, such as porcine epidemic diarrhea virus (PEDv), scotophilus bat coronavirus 512, bat coronavirus CDPHE15, BtRF-Alpha-CoV HuB-2013, bat coronavirus HKU10, miniopterus bat coronavirus HKU8, miniopterus bat coronavirus 1, Nyctalus velutinus alphacoronavirus SC-2013, Pipistrellus kuhli coronavirus 3398, Myotis ricketti alphacoronavirus Sax-2011, HumCoV 229E, 229E-related bat coronavirus, camel alphacoronavirus, alpaca respiratory coronavirus, HumCoV NL63, NL63-related Bat-CoV BtKYNL63-9b, HKU2, SADSr-CoV, SADS-CoV, Lucheng Rn rat coronavirus2, FIPV, TGEV, PRCV, alphacoronavirus 1, mink coronavirus 1, FRCoV-NL-2010, Sorex araneus coronavirus T14, Suncus murinus coronavirus X74, HumCoV OC43, HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYNO2, human SARS-CoV-2, cat-CoV, tiger-CoV, dog-CoV, mink-CoV, GD pangolin-CoV, bat Hp-betacoronavirus Zhejiang2013, peafowl IBV, avian coronavirus 9203, fowl IBV, avian coronavirus, duck-CoV, partridge IBV, goose coronavirus CB17, beluga whale coronavirus SW1, HKU22, HKU19, HKU20, HKU21, HKU11, HKU12, HKU16, HKU27, HKU28, HKU29, HKU18, HKU13, HKU30-UAE, HKU30-Poland, HKU17-USA, HKU17-China, AlCCoV, and HKU15, HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYNO2, human SARS-CoV-2, cat-CoV, tiger-CoV, dog-CoV, mink-CoV, GD pangolin-CoV, and bat Hp-betacoronavirus Zhejiang2013, or combinations thereof. A pharmaceutical composition containing an effective amount of any compound described herein is administered to the patient, such as via a nebulizer. The patient is monitored until symptoms are alleviated or ameliorated, and the pharmaceutical composition may be administered one or more additional times if it is determined that such administration is necessary or helpful for treatment.

### Example 16

### Treatment of Human Patient Having a Lung Infection Arising from Cystic Fibrosis

A human patient is identified as having a lung infection arising from cystic fibrosis. A pharmaceutical composition containing an effective amount of any compound described herein is administered to the patient, such as via a nebulizer. The patient is monitored until symptoms are alleviated or ameliorated, and the pharmaceutical composition may be administered one or more additional times if it is determined that such administration is necessary or helpful for treatment.

### Example 17

### Treatment of Human Patient Having Pneumonia

A human patient is identified as having pneumonia. A pharmaceutical composition containing an effective amount of any compound described herein is administered to the patient, such as via a nebulizer. The patient is monitored until symptoms are alleviated or ameliorated, and the pharmaceutical composition may be administered one or more additional times if it is determined that such administration is necessary or helpful for treatment.

### Example 18

### Treatment of Human Patient Having Ventilator Acquired Pneumonia

A human patient is identified as having ventilator acquired pneumonia. A pharmaceutical composition containing an effective amount of any compound described herein is administered to the patient, such as via a nebulizer. The patient is monitored until symptoms are alleviated or ameliorated, and the pharmaceutical composition may be administered one or more additional times if it is determined that such administration is necessary or helpful for treatment.

### Example 19

### Treatment of Human Patient Having an Infection in a Burn Wound

A human patient is identified as having an infection in a burn wound. A pharmaceutical composition containing an effective amount of any compound described herein is administered to the patient, such as topically at the location of the burn wound. The patient is monitored until symptoms are alleviated or ameliorated, and the pharmaceutical composition may be administered one or more additional times if it is determined that such administration is necessary or helpful for treatment.

### Example 20

### Treatment of Human Patient Having Otitis Externa

A human patient is identified as having otitis externa. A pharmaceutical composition containing an effective amount of any compound described herein is administered, such as topically directly into the patient's external ear canal. The patient is monitored until symptoms are alleviated or ameliorated, and the pharmaceutical composition may be administered one or more additional times if it is determined that such administration is necessary or helpful for treatment.

### Example 21

### Treatment of Female Human Patient Having Bacterial Vaginosis

A female human patient is identified as having bacterial vaginosis. A pharmaceutical composition containing an effective amount of any compound described herein is administered, such as topically onto or into the patient's vagina. The patient is monitored until symptoms are alleviated or ameliorated, and the pharmaceutical composition may be administered one or more additional times if it is determined that such administration is necessary or helpful for treatment.

### Example 22

### Treatment of Human Patient Having Impetigo

A human patient is identified as having impetigo. A pharmaceutical composition containing an effective amount of any compound described herein is administered, such as topically to the patient at the location of the vesicles, pustules and/or yellowish crusts. The patient is monitored until symptoms are alleviated or ameliorated, and the pharmaceutical composition may be administered one or more additional times if it is determined that such administration is necessary or helpful for treatment.

### Example 23

### Treatment of Human Patient Having Oral Mucositis

A human patient is identified as having oral mucositis. A pharmaceutical composition containing an effective amount of any compound described herein is administered, such as topically directly into the patient's external ear canal. The patient is monitored until symptoms are alleviated or ameliorated, and the pharmaceutical composition may be administered one or more additional times if it is determined that such administration is necessary or helpful for treatment.

While embodiments have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

1. A compound having the formula: 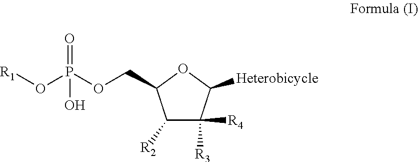
or a pharmaceutically acceptable salt thereof, wherein
R₁ is (C₁-C₈)straight-chain alkyl, (C₃-C₈)branched-chain alkyl, (C₃-C₈)cycloalkyl, phenylalkyl, alkylcycloalkyl, hydroxy(C₁-C₈)straight-chain alkyl, hydroxy(C₃-C₈)branched-chain alkyl, alkoxy(C₁-C₈)straight-chain alkyl, alkoxy(C₃-C₈)branched-chain alkyl, halo(C₁-C₈)straight-chain alkyl, or halo(C₃-C₈)branched-chain alkyl;
R₂, R₃, and R₄ are each independently H, OH, oxy(C₁-C₈)straight-chain alkyl, oxy(C₁-C₈)branched-chain alkyl, CF₃, or F;
Heterobicycle is selected from the group consisting of 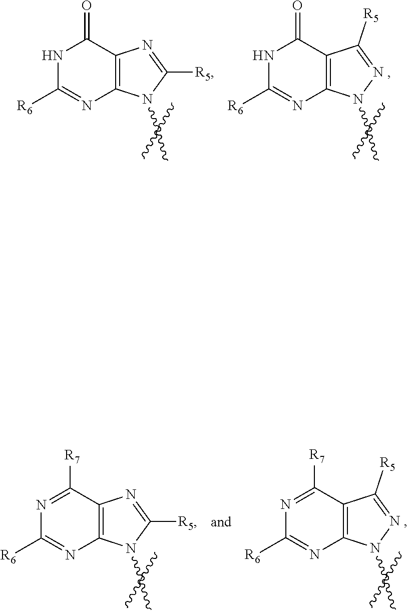
R₅ is H, (C₁-C₅)straight-chain alkyl, (C₁-C₅)branched-chain alkyl, or CF₃;
R₆ and R₇ are each independently selected from H, N(R₈)R₉, (C₁-C₅)straight-chain alkyl, (C₁-C₅)branched-chain alkyl, or CF₃; and
R₈ and R₉ are each independently selected from H, (C₁-C₅)straight-chain alkyl, (C₁-C₅)branched-chain alkyl, or are bound to one another through a C—C bond to form a C₃-C₈ cycloalkyl.

2. The compound according to claim 1, wherein R₁ is octyl.

3. The compound according to claim 1, wherein R₁ is hexyl.

4. The compound according to claim 1, wherein R₁ is butyl.

5. The compound according to claim 1, wherein R₁ is 2-methylpropyl.

6. The compound according to claim 1, wherein R₁ is 2-methylbutyl.

7. The compound according to claim 1, wherein R₁ is 1-butanolyl.

8. The compound according to claim 1, wherein R₂ is H.

9. The compound according to claim 1, wherein R₂ is OH.

10. The compound according to claim 1, wherein R₃ and R₄ are both H.

11. The compound according to claim 1, wherein R₃ and R₄ are both F.

12. The compound according to claim 1, wherein R₃ is OCH₃ and R₄ is H.

13. The compound according to claim 1, wherein R₃ is H and R₄ is OCH₃.

14. The compound according to claim 1, wherein Heterobicycle is

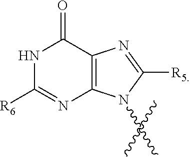

15. The compound according to claim 14, wherein R₅ is H and R₆ is NH₂.

16. The compound according to claim 15, wherein R₃ is OCH₃.

17. The compound according to claim 1, wherein Heterobicycle is

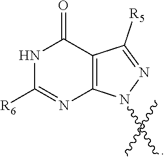

18. The compound according to claim 1, wherein Heterobicycle is

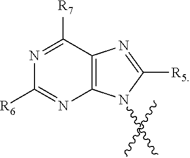

19. The compound according to claim 18, wherein R₅ and R₆ are both H and R₇ is NH₂.

20. The compound according to claim 18, wherein R₅ and R₇ are both H and R₆ is NH₂.

21. The compound according to claim 1, wherein Heterobicycle is

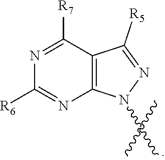

22. The compound according to claim 21, wherein R₅ and R₆ are both H and R₇ is NH₂.

23. A compound having the formula: 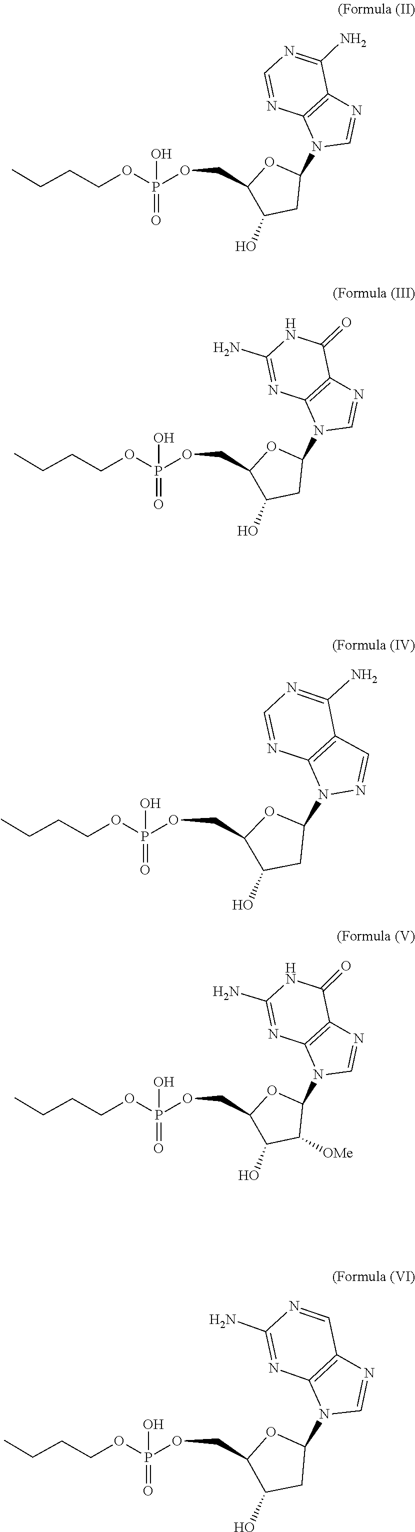
and a pharmaceutically acceptable salt thereof.

24. (canceled)

25. (canceled)

26. (canceled)

27. (canceled)

28. A pharmaceutical composition comprising the compound according to claim 1, or a pharmaceutically acceptable salt thereof, and one or more pharmaceutically acceptable carriers, excipients, diluents, or a mixture of two or more thereof.

29. A method of treating an infection in a patient in need thereof, the method comprising administering an effective amount of a compound according to claim 1, or a pharmaceutically acceptable salt thereof, to the patient.

30. A method of treating an infection of at least one of a wound or an ulcer in a patient in need thereof, the method comprising administering an effective amount of a compound according to claim 1, or a pharmaceutically acceptable salt thereof, to the patient.

31. A method of treating an infection of a diabetic foot ulcer in a patient in need thereof, the method comprising administering an effective amount of a compound according to claim 1, or a pharmaceutically acceptable salt thereof, to the patient.

32. A method of treating a bladder and/or a urinary tract infection in a patient in need thereof, the method comprising administering an effective amount of a compound according to claim 1, or a pharmaceutically acceptable salt thereof, to the patient.

33. A method of treating a lung infection in a patient in need thereof, the method comprising administering an effective amount of a compound according to claim 1, or a pharmaceutically acceptable salt thereof, to the patient.

34. The method of claim 33, wherein the lung infection arises from a pulmonary condition.

35. The method of claim 34, wherein the pulmonary condition is selected from the group consisting of genetic conditions, acquired conditions, primary conditions, secondary conditions, asthma, chronic obstructive pulmonary disease, cystic fibrosis, bronchiolitis, pneumonia, bronchitis, emphysema, adult respiratory distress syndrome, allergies, lung cancer, small cell lung cancer, primary lung cancer, metastatic lung cancer, bronchiestasis, bronchopulmonary dysplasia, chronic bronchitis, chronic lower respiratory diseases, croup, high altitude pulmonary edema, pulmonary fibrosis, interstitial lung disease, reactive airway disease, lymphangioleiomyomatosis, neonatal respiratory distress syndrome, parainfluenza, pleural effusion, pleurisy, pneumothorax, primary pulmonary hypertension, psittacosis, pulmonary edema secondary to various causes, pulmonary embolism, pulmonary hypertension secondary to various causes, respiratory failure secondary to various causes, sleep apnea, sarcoidosis, smoking, stridor, acute respiratory distress syndrome, infectious diseases, SARS, tuberculosis, psittacosis infection, Q fever, parainfluenza, respiratory syncytial virus, combinations thereof, and conditions caused by any one or combination of the above.

36. The method of claim 34, wherein the pulmonary condition arises from a coronavirus.

37. The method of claim 36, wherein the coronavirus is selected from the group consisting of an alphacoronavirus, a betacoronavirus, a gammacoronavirus, a deltacoronavirus, and an omicroncoronavirus, or combinations thereof.

38. The method of claim 36, wherein the coronavirus is selected from the group consisting of porcine epidemic diarrhea virus (PEDv), scotophilus bat coronavirus 512, bat coronavirus CDPHE15, BtRF-Alpha-CoV HuB-2013, bat coronavirus HKU10, miniopterus bat coronavirus HKU8, miniopterus bat coronavirus 1, Nyctalus velutinus alphacoronavirus SC-2013, Pipistrellus kuhli coronavirus 3398, Myotis ricketti alphacoronavirus Sax-2011, HumCoV 229E, 229E-related bat coronavirus, camel alphacoronavirus, alpaca respiratory coronavirus, HumCoV NL63, NL63-related Bat-CoV BtKYNL63-9b, HKU2, SADSr-CoV, SADS-CoV, Lucheng Rn rat coronavirus2, FIPV, TGEV, PRCV, alphacoronavirus 1, mink coronavirus 1, FRCoV-NL-2010, Sorex araneus coronavirus T14, Suncus murinus coronavirus X74, HumCoV OC43, HumCoV OC43 isolate TNP F1778 2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYNO2, human SARS-CoV-2, cat-CoV, tiger-CoV, dog-CoV, mink-CoV, GD pangolin-CoV, bat Hp-betacoronavirus Zhejiang2013, peafowl IBV, avian coronavirus 9203, fowl IBV, avian coronavirus, duck-CoV, partridge IBV, goose coronavirus CB17, beluga whale coronavirus SW1, HKU22, HKU19, HKU20, HKU21, HKU11, HKU12, HKU16, HKU27, HKU28, HKU29, HKU18, HKU13, HKU30-UAE, HKU30-Poland, HKU17-USA, HKU17-China, AlCCoV, and HKU15, or combinations thereof.

39. The method of claim 36, wherein the coronavirus is a betacoronavirus selected from the group consisting of HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYNO2, human SARS-CoV-2, cat-CoV, tiger-CoV, dog-CoV, mink-CoV, GD pangolin-CoV, and bat Hp-betacoronavirus Zhejiang2013, or combinations thereof.

40. A method of treating cystic fibrosis in a patient in need thereof, the method comprising administering an effective amount of a compound according to claim 1, or a pharmaceutically acceptable salt thereof, to the patient.

41. A method of treating pneumonia in a patient in need thereof, the method comprising administering an effective amount of a compound according to claim 1, or a pharmaceutically acceptable salt thereof, to the patient.

42. The method of claim 41, wherein the pneumonia is ventilator acquired pneumonia.

43. A method of treating an infection in a burn wound in a patient in need thereof, the method comprising administering an effective amount of a compound according to claim 1, or a pharmaceutically acceptable salt thereof, to the patient.

44. A method of treating otitis externa in a patient in need thereof, the method comprising administering an effective amount of a compound according to claim 1, or a pharmaceutically acceptable salt thereof, to the patient.

45. A method of treating bacterial vaginosis in a patient in need thereof, the method comprising administering an effective amount of a compound according to claim 1, or a pharmaceutically acceptable salt thereof, to the patient.

46. A method of treating impetigo in a patient in need thereof, the method comprising administering an effective amount of a compound according to claim 1, or a pharmaceutically acceptable salt thereof, to the patient.

47. A method of treating oral mucositis in a patient in need thereof, the method comprising administering an effective amount of a compound according to claim 1, or a pharmaceutically acceptable salt thereof, to the patient.
